(19) United States
(12) Patent Application Publication
(10) Pub. No.: US 2022/0273757 A1
(43) Pub. Date: Sep. 1, 2022

(54) METHODS FOR ADMINISTERING ANGIOTENSIN II BY MONITORING RENIN LEVELS

(71) Applicant: La Jolla Pharma, LLC, San Diego, CA (US)

(72) Inventor: Lakhmir Chawla, San Diego, CA (US)

(21) Appl. No.: 17/618,343

(22) PCT Filed: Jun. 12, 2020
(86) PCT No.: PCT/US2020/037524

§ 371 Date: Dec. 10, 2021

Related U.S. Application Data
(60) Provisional application No. 62/878,233, filed on Jul. 24, 2019.
(60) Provisional application No. 62/860,976, filed on Jun. 13, 2019.

(51) Int. Cl. A61K 38/08; A61K 38/095; A61P 9/02; A61K 9/00; A61K 31/401; A61K 31/403; A61K 31/675

(52) U.S. Cl. CPC A61K 38/085; A61K 38/095; A61P 9/02; A61K 9/0019; A61K 31/401; A61K 31/403; A61K 31/675

(57) ABSTRACT

The present disclosure relates to the use of angiotensin II in therapeutic methods for the treatment of hypotension.

### RELATED APPLICATIONS

This application claims priority to U.S. Provisional Application No. 62/860,976, filed Jun. 13, 2019, and U.S. Provisional Application No. 62/878,233, filed Jul. 24, 2019, each of which is incorporated herein by reference in its entirety.

### BACKGROUND

Hypotension, if uncorrected, is life-threatening and occurs as the result of various underlying conditions such as trauma, septic shock or drug reactions. The first line of treatment is intravenous fluids, and if this fails to correct the hypotension then vasopressors are deployed. The first line vasopressor is a catecholamine infusion. Catecholamines are amines derived from the amino acid tyrosine, and they include epinephrine (adrenaline), norepinephrine (noradrenaline), phenylephrine, and dopamine, which act as both hormones and neurotransmitters that increase blood pressure. While largely effective at treating hypotension, some patients fail to respond to adequate doses and are defined as catecholamine-resistant. These patients frequently have a high mortality and no acceptable alternatives. Hypotension can also lead to life-threatening conditions, such as acute kidney injury (AKI).

Thus, alternate methods of regulating blood pressure in patients with hypotension are needed.

### SUMMARY

Angiotensin II is a peptide hormone naturally produced by the body that regulates blood pressure via vasoconstriction and sodium reabsorption. The hemodynamic effects of angiotensin II administration have been the subject of numerous clinical studies, demonstrating significant effects on systemic and renal blood flow. The invention disclosed herein relates to methods of suppressing blood renin plasma or serum levels, suppressing plasma renin activity (PRA), treating hypotension, AKI or shock in a subject or other indications by measuring renin levels in a subject and administering angiotensin II to the subject. Suppression of renin and PRA may be useful in patients with higher than normal levels of renin, or in patients with normal or typical amounts of renin but who would still benefit from lowering renin levels (e.g., patients with any indication disclosed herein). Also provided herein are methods of identifying patients that would benefit from renin suppression (e.g., patients with any indication disclosed herein) by measuring renin levels in such patients.

Various aspects of the invention relate, in part, to the finding that renin levels in individuals is suppressible and angiotensin II can lower renin levels. Additionally, the invention relates, in part, to the treatment of hypotension and other indications disclosed herein by first determining whether an individual would be a candidate for angiotensin therapy based on the patient's measured renin, PRA, or prorenin levels. Additionally, renin levels can inform a medical practitioner of the appropriate angiotensin II dosage regimen. Administration of angiotensin II can treat hypotension, and the levels of renin may indicate that a patient would be a candidate for angiotensin II administration. In some embodiments, the patient has high renin levels but does not have hypotension. Lower doses of angiotensin II therapy may be administered to hypotensive patients who are predicted to be sensitive to angiotensin II therapy relative to other hypotensive patients, e.g., to inhibit or prevent undesired side effects of exogenous angiotensin II, such as hypertension.

In some aspects, the invention relates to methods of treating hypotension, such as catecholamine-resistant hypotension, in a patient in need thereof, comprising administering to the patient angiotensin II. The term “catecholamine-resistant hypotension” as used herein refers to patients who require more than 15 μg/kg/min of dopamine, 0.1 μg/kg/min norepinephrine, or 0.1 μg/kg/min epinephrine as a vasopressor. Dopamine, norepinephrine, and epinephrine may be administered at rates higher than 15 μg/kg/min, 0.1 μg/kg/min, or 0.1 μg/kg/min, respectively, but elevated rates correlate with increased mortality.

In some aspects, provided herein are methods of treating hypotension, treating acute kidney injury, acute respiratory distress syndrome (ARDS), hepatorenal syndrome, cirrhosis, and/or suppressing plasma renin activity (PRA) or renin levels in a human patient by measuring a renin level in the patient, thereby obtaining an initial renin level; and if the initial renin level is at least a threshold value, administering to the patient angiotensin II at an initial rate. The initial renin level threshold value may 50 pg/mL, 100 pg/mL, 150 pg/mL, 200 pg/mL, or 300 pg/mL.

In some aspects, provided herein are methods of treating hypotension, treating or preventing AKI, ARDS, hepatorenal syndrome, cirrhosis, and/or suppressing PRA or renin levels (e.g., blood renin plasma or serum levels) in a human patient by measuring an initial PRA level in the patient, thereby obtaining an initial PRA level; and if the initial PRA is at least a threshold value, administering to the patient angiotensin II at an initial rate. The initial PRA threshold value may be 0.6 ng/mL/hour, 1.0 ng/mL/hour, 1.5 ng/mL/hour, 2.0 ng/mL/hour, 2.5 ng/mL/hour, 3.0 ng/mL/hour, 3.5 ng/mL/hour, 4.0 ng/mL/hour, 4.5 ng/mL/hour, 5.0 ng/mL/hour, 5.5 ng/mL/hour, 6.0 ng/mL/hour, 7 ng/mL/hour, 8 ng/mL/hour, 9 ng/mL/hour, 10 ng/mL/hour, 15 ng/mL/hour, or 20 ng/mL/hour.

In some embodiments, the human patient's MAP (i.e., MAP prior to angiotensin II administration) is at least 45 mm Hg, at least 55 mm Hg, at least 65 mm Hg, at least 75 mmHg, or at least 85 mm Hg.

In some embodiments, the patient does not have hypotension, shock, sepsis, ARDS, AKI, hepatorenal syndrome, or cirrhosis. The patient may be at risk for hypotension, shock, sepsis, ARDS, AKI, hepatorenal syndrome, and/or cirrhosis. In some embodiments, the patient is afflicted with hypotension, shock, sepsis, ARDS, AKI, hepatorenal syndrome, and/or cirrhosis.

The patient may have elevated renin shock, sepsis, septic shock, distributive shock, or cardiogenic shock. In some embodiments, the patient does not have elevated renin shock, sepsis, septic shock, distributive shock, or cardiogenic shock. In some embodiments, the patient is at risk for elevated renin shock, sepsis, septic shock, distributive shock, or cardiogenic shock.

The initial rate of angiotensin II may be at least 0.1 ng/kg/min, at least 0.5 ng/kg/min, at least 1 ng/kg/min, at least 2.5 ng/kg/min, at least 5 ng/kg/min, at least 10 ng/kg/min, at least 20 ng/kg/min of angiotensin II. In some embodiments, the initial rate is less than 1 ng/kg min, less than 2.5 ng/kg/min, less than 5 ng/kg/min, or less than 10 ng/kg/min. The patient may have acute kidney injury.

As described herein, administering angiotensin II can prevent an increase in blood renin levels, which in turn can prevent AKI, lessen the severity of any hypotension, reduce needs for fluid administration and improve clinical outcomes. Angiotensin can be used to prevent renin increase in connection with cardiac surgery or non-cardiac surgery, and for early treatment in cardiogenic shock or vasodilatory shock.

In some aspects, provided herein are methods of treating hypotension, treating AKI, acute respiratory distress syndrome (ARDS), hepatorenal syndrome, cirrhosis and/or suppressing renin or PRA levels in a human patient, by measuring a renin level in the patient, thereby obtaining an initial renin level; and if the initial renin level is at least 2× (e.g., at least 3×, at least 4×, at least 5×, at least 6×, at least 7×, at least 8×, at least 9×, or at least 10×) the value of a normal renin level, administering to the patient angiotensin II at an initial rate. The normal renin level may be 5 ng/ml-55 ng/ml. The normal renin level may be calculated by any method disclosed herein. The method may further comprise administering angiotensin II according to the methods disclosed herein until the subject's renin level is less than 2×, less than 3×, less than 4×, less than 5×, less than 6×, less than 7×, less than 8×, less than 9×, or less than 10× the value of the normal renin level.

The patient may be receiving or has received an angiotensin converting enzyme inhibitor (ACE inhibitor) (e.g., perindopril, captopril, enalapril, lisinopril, benazepril, fosinopril, moexipril, quinapril, trandolapril, and Ramipril). The patient may have ACE dysfunction. In some embodiments, the ACE activity in the patient is or has been inhibited or is deficient. For example, a patient may have endothelial injury or a genetic mutation which causes ACE dysfunction. The patient may have endothelial injury. The patient may be receiving renal replacement therapy.

The method may comprise increasing the rate of angiotensin II administration prior to obtaining a second measurement of renin level or PRA. The method may comprise measuring the patient's renin level or PRA after a period of time, and if the patient's measured renin level or PRA is less than a threshold value, decreasing the rate of administration of angiotensin II. In some embodiments, the method comprises measuring the patient's renin level after a period of time, and if the patient's measured renin level or PRA is at least a threshold value, increasing or maintaining the rate of administration of angiotensin II. The renin level threshold value may be 50 pg/mL, 100 pg/mL, 150 pg/mL, 200 pg/mL, or 300 pg/mL. The initial PRA threshold value may be 0.6 ng/mL/hour, 1.0 ng/mL/hour, 1.5 ng/mL/hour, 2.0 ng/mL/hour, 2.5 ng/mL/hour, 3.0 ng/mL/hour, 3.5 ng/mL/hour, 4.0 ng/mL/hour.

In some embodiments, the method may comprise measuring the patient's renin level after a period of time, and if the difference between the patient's initial renin level and the measured renin level is at least 5 pg/ml, at least 10 pg/ml, at least 25 pg/ml, at least 30 pg/ml, at least 35 pg/ml, at least 40 pg/ml, at least 45 pg/ml, at least 50 pg/ml, at least 55 pg/ml, at least 60 pg/ml, at least 65 pg/ml, at least 70 pg/ml, at least 75 pg/ml, at least 80 pg/ml, at least 85 pg/ml, at least 90 pg/ml, at least 95 pg/ml, at least 100 pg/ml, at least 110 pg/ml, at least 120 pg/ml, at least 130 pg/ml, at least 140 pg/ml, at least 150 pg/ml, at least 160 pg/ml, at least 170 pg/ml, at least 180 pg/ml, at least 190 pg/ml, at least 200 pg/ml, at least 210 pg/ml, at least 220 pg/ml, at least 230 pg/ml, at least 240 pg/ml, at least 250 pg/ml, at least 260 pg/ml, at least 270 pg/ml, at least 280 pg/ml, at least 290 pg/ml, at least 300 pg/ml, at least 310 pg/ml, at least 320 pg/ml, at least 330 pg/ml, at least 340 pg/ml, 350 pg/ml, at least 360 pg/ml, at least 370 pg/ml, at least 380 pg/ml, at least 390 pg/ml, at least 400 pg/ml, at least 410 pg/ml, at least 420 pg/ml, at least 430 pg/ml, at least 440 pg/ml, or at least 450 pg/ml, maintaining or decreasing the rate of administration of angiotensin II.

In some embodiments, the method further comprises measuring the patient's renin level after a period of time, and if the difference between the patient's initial renin level and the patient's measured renin level is less than or equal to 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, 200 pg/ml, 225 pg/ml, 250 pg/ml, 275 pg/ml, 300 pg/ml, 325 pg/ml, 350 pg/ml, 375 pg/ml, 400 pg/ml, 425 pg/ml, 450 pg/ml, 475 pg/ml, or 500 pg/ml, increasing or maintaining the rate of administration of angiotensin II.

In some embodiments, the method may comprise measuring the patient's PRA level after a period of time, and if the difference between the patient's initial patient's PRA level and the measured patient's PRA level is at least 0.01 ng/mL/hour, at least 0.05 ng/mL/hour, at least 0.1 ng/mL/hour, at least 0.2 ng/mL/hour, at least 0.3 ng/mL/hour, at least 0.4 ng/mL/hour, at least 0.5 ng/mL/hour, at least 0.6 ng/mL/hour, at least 0.7 ng/mL/hour, at least 0.8 ng/mL/hour, at least 0.9 ng/mL/hour, at least 1.0 ng/mL/hour, at least 1.1 ng/mL/hour, at least 1.2 ng/mL/hour, at least 1.3 ng/mL/hour, at least 1.4 ng/mL/hour, at least 1.5 ng/mL/hour, at least 1.6 ng/mL/hour, at least 1.7 ng/mL/hour, at least 1.8 ng/mL/hour, at least 1.9 ng/mL/hour, at least 2.0 ng/mL/hour, at least 2.2 ng/mL/hour, at least 2.4 ng/mL/hour, at least 2.6 ng/mL/hour, at least 2.8 ng/mL/hour, at least 3.0 ng/mL/hour, at least 3.2 ng/mL/hour, at least 3.4 ng/mL/hour, at least 3.6 ng/mL/hour, at least 3.8 ng/mL/hour, at least 4.0 ng/mL/hour, least 5.0 ng/mL/hour, or at least 10 ng/mL/hour, maintaining or decreasing the rate of administration of angiotensin II.

In some embodiments, the method comprises measuring the patient's PRA level after a period of time, and if the difference between the patient's initial PRA level and the patient's measured PRA level is less than or equal to 0.0001 ng/mL/hour, 0.001 ng/mL/hour, 0.005 ng/mL/hour, 0.01 ng/mL/hour, 0.02 ng/mL/hour, 0.03 ng/mL/hour, 0.04 ng/mL/hour, 0.05 ng/mL/hour, 0.06 ng/mL/hour, 0.07 ng/mL/hour, 0.08 ng/mL/hour, 0.09 ng/mL/hour, 0.1 ng/mL/hour, 0.2 ng/mL/hour, 0.5 ng/mL/hour, 0.7 ng/mL/hour, 0.9 ng/mL/hour, 1.0 ng/mL/hour, 1.2 ng/mL/hour, 1.3 ng/mL/hour, 1.5 ng/mL/hour, 2.0 ng/mL/hour, 2.5 ng/mL/hour, 3.0 ng/mL/hour, 3.5 ng/mL/hour, 5 ng/mL/hour, 7 ng/mL/hour, 10 ng/mL/hour, or 15 ng/mL/hour, increasing or maintaining the rate of administration of angiotensin II.

In some embodiments, the methods provided herein further comprise measuring the patient's mean arterial pressure after a period of time, and if the measured mean arterial pressure is at least 55 mm Hg, 60 mm Hg, 65 mm Hg, 70 mm Hg, 75 mm Hg, or 80 mm Hg, decreasing the rate of administration of angiotensin II. In some embodiments, the method further comprises measuring the patient's mean arterial pressure after a period of time, and if the measured mean arterial pressure is below 55 mm Hg, 60 mm Hg, 65 mm Hg, 70 mm Hg, 75 mm Hg, or 80 mm Hg, increasing or maintaining the rate of administration of angiotensin II.

The period of time may be less than one hour, about one hour, about two hours, about three hours, about four hours, about five hours, about six hours, about 10 hours, about 12 hours, about 14 hours, about 16 hours, about 18 hours, about 20 hours, about 22 hours, about 24 hours, or about 48 hours.

In some embodiments, the patient is receiving a vasopressor prior to administering angiotensin II. In some embodiments, the angiotensin II is administered continuously for at least 1 hour, at least 6 hours, at least 10 hours, at least 12 hours, at least 16 hours, at least 20 hours, at least 24 hours, at least 48 hours, at least 72 hours, or at least 5 days. In some embodiments, the angiotensin II is administered for no more than 7 days.

In some embodiments, the renin level in the human patient after angiotensin administration is less than 1000 pg/mL, less than 900 pg/mL, less than 800 pg/mL, less than 700 pg/mL, less than 600 pg/mL, less than 500 pg/mL, less than 400 pg/mL, less than 300 pg/mL, less than 200 pg/mL, less than 150 pg/mL, less than 100 pg/mL, or less than 50 pg/mL.

In some embodiments, the PRA level in the human patient after angiotensin administration is less than 1.5 ng/mL/hour, 2.0 ng/mL/hour, 2.5 ng/mL/hour, 3.0 ng/mL/hour, 3.5 ng/mL/hour, 4 ng/mL/hour, 5 ng/mL/hour, 10 ng/mL/hour, 20 ng/mL/hour, or 30 ng/mL/hour. In some embodiments, the patient has an initial mean arterial pressure of 70 mm Hg, 65 mm Hg, 60 mm Hg, or 55 mm Hg or less prior to administering the composition. In some aspects, provided herein are methods of determining whether a patient afflicted with hypotension and/or shock (e.g., elevated renin shock) would be a candidate for angiotensin II therapy. The method may comprise measuring a renin level in the patient, thereby obtaining an initial renin level, and if the initial renin level is at least a threshold value, the patient is a candidate for angiotensin II therapy. The method may further include administering to the patient angiotensin II at an initial rate. The threshold value may be 100 pg/mL, 150 pg/mL, 200 pg/mL, or 300 pg/mL. The threshold value may be a multiple of the value of a normal or mean renin level. For example, the threshold value may be at least 3×, at least 4×, or at least 5× the value of a normal renin level. The method may further comprise administering angiotensin II until the patient's renin level is less than 5×, less than 4×, less than 3× or less than 2× the value of a normal renin level.

In some aspects, provided herein are methods of suppressing renin in patients in need thereof that do not have hypotension by administering angiotensin II according to the methods provided herein. Therefore, in some embodiments, the patient does not have sepsis, septic shock, distributive shock, cardiogenic shock, ARDS, hepatorenal syndrome, or cirrhosis. In some embodiments, the patient does not have sepsis, septic shock, distributive shock, cardiogenic shock, acute respiratory distress syndrome (ARDS), hepatorenal syndrome, AKI, cirrhosis, but does have elevated renin levels (e.g., blood renin plasma or serum levels). In some embodiments, the patient is at risk for developing sepsis, septic shock, distributive shock, cardiogenic shock, AKI, acute respiratory distress syndrome (ARDS), hepatorenal syndrome, or cirrhosis or any other indication disclosed herein. For example, the patient may be recovering from surgery but has not developed symptoms associated with hypotension, sepsis, septic shock, distributive shock, cardiogenic shock, AKI, acute respiratory distress syndrome (ARDS), hepatorenal syndrome, cirrhosis. In other embodiments, the patient may be recovering from surgery but has developed symptoms associated with hypotension, sepsis, septic shock, distributive shock, cardiogenic shock, AKI, acute respiratory distress syndrome (ARDS), hepatorenal syndrome, cirrhosis.

### BRIEF DESCRIPTION OF THE FIGURES

FIG. 1 shows day 28 mortality by baseline renin.

FIG. 2 shows day 28 mortality in patients receiving angiotensin II who had an initial renin level that was greater than the patient population median.

FIG. 3 shows renin-angiotensin-aldosterone system disturbance hypothesis. ACE, angiotensin-converting enzyme.

FIGS. 4A and 4B show serum angiotensin I and renin levels. FIG. 4A shows serum angiotensin I levels. FIG. 4B shows serum renin levels. All values are median (interquartile range).

FIGS. 5A and 5B shows a Kaplan-Meier survival plot according to renin levels and treatment with angiotensin II or placebo. (A) Day 28 survival: renin level below population median. (B) Day 28 survival: renin level above population median.

FIG. 6 shows cubic splines of mortality and baseline serum renin.

FIGS. 7A, 7B, 7C, and 7D shows Day-28 survival by treatment arm by 1×ULN (FIG. 7A), 2×ULN (FIG. 7B), 3×ULN (FIG. 7C), and 4×ULN (FIG. 7D).

FIGS. 8A, 8B, 8C, and 8D shows Day-28 survival by treatment arm by first quartile (FIG. 8A), second quartile (FIG. 8B), third quartile, (FIG. 8C), and forth quartile (FIG. 8D).

FIG. 9 shows survival by treatment arm and renin level.

FIG. 10 shows renal recovery in patients with severe AKI treatment by assignment and renin status.

FIG. 11 shows renin levels in the subset populations of ATHOS-3 trial.

### DETAILED DESCRIPTION

### General

Provided herein are methods of treating hypotension, suppressing renin levels (e.g., blood renin plasma or serum levels), and/or treating AKI in a human patient by measuring renin levels in the patient, thereby obtaining an initial measurement of renin levels in the patient; and if the renin levels are at least a threshold value, and administering to the patient angiotensin II at an initial rate. In some aspects, provided herein are methods of determining whether an individual is a candidate for angiotensin therapy by measuring renin levels in the patient, thereby obtaining an initial measurement of renin levels in the patient; and if the renin levels are at least a threshold value, selecting the individual for angiotensin II administration.

In some aspects, suppressing renin levels (e.g., blood renin plasma or serum levels) in patients may be beneficial to any patient that has elevated renin levels and/or any patient at risk for or afflicted with an indication disclosed herein. The patient may not be showing signs or symptoms of hypotension or hypotension-related disorders or diseases (e.g., shock, or any other indication disclosed herein), but may nevertheless benefit from suppressing renin levels. Patients who are at risk for developing or afflicted with elevated renin, shock, hypotension, AKI, acute respiratory distress syndrome (ARDS), hepatorenal syndrome, or cirrhosis may benefit from suppressing renin levels, even if the patient's renin levels are not elevated.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “about” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “administering” means providing a pharmaceutical agent or composition to a subject, and includes, but is not limited to, administering by a medical professional and self-administering. Such an agent, for example, may be hepcidin or a hepcidin analogue.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, a therapeutic that “prevents” a condition (e.g., iron overload) refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

In certain embodiments, agents of the invention may be used alone or conjointly administered with another type of therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

As used herein, the term “subject” means a human or non-human animal selected for treatment or therapy. As used herein, the term patient and subject are interchangeable.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Methods

Provided herein are methods of suppressing renin levels (e.g., blood renin plasma or serum levels) in a patient in need thereof. In some embodiments, the patient is hypotensive. In other embodiments, the patient is not hypotensive. The patient may have an initial MAP (i.e., MAP prior to administration of angiotensin II) of at least 55 mm Hg, at least 65 mmHg, or at least 75 mmHg. Provided herein are methods of suppressing plasma renin activity (PRA) in patient in need thereof. Also provided herein are methods of treating hypotension, AKI, and/or shock in patients by measuring renin levels and/or measuring the patient's PRA in the patient, thereby obtaining an initial measurement of renin levels and/or PRA (referred to as an initial measurement of renin levels or initial measurement of PRA herein, which is distinguished from a measured renin level or measured PRA that is determined after administering angiotensin II to the patient) in the patient and administering to the patient angiotensin II at an initial rate if the renin levels or PRA are at least a threshold value. In some aspects, provided herein are methods of determining whether an individual is a candidate for angiotensin therapy by measuring renin levels and/or PRA in the patient, thereby obtaining an initial measurement of renin levels and/or PRA in the patient; and if the renin levels and/or PRA levels are at least a threshold value, selecting the individual for angiotensin II administration.

Also provided herein are methods of treating hypotension, AKI, and/or shock in patients by measuring prorenin levels in the patient, thereby obtaining an initial measurement of prorenin levels (referred to as an initial measurement of prorenin levels, which is distinguished from a measured prorenin level that is determined after administering angiotensin II to the patient) in the patient and administering to the patient angiotensin II at an initial rate if the prorenin levels are less than 5 pg/ml, less than 10 pg/ml, less than 25 pg/ml, less than 30 pg/ml, less than 35 pg/ml, less than 40 pg/ml, less than 45 pg/ml, less than 50 pg/ml, less than 55 pg/ml, less than 60 pg/ml, less than 65 pg/ml, less than 70 pg/ml, less than 75 pg/ml, less than 80 pg/ml, less than 85 pg/ml, less than 90 pg/ml, less than 95 pg/ml, less than 100 pg/ml, less than 110 pg/ml, less than 120 pg/ml, less than 130 pg/ml, less than 140 pg/ml, less than 150 pg/ml, less than 160 pg/ml, less than 170 pg/ml, less than 180 pg/ml, less than 190 pg/ml, less than 200 pg/ml, less than 210 pg/ml, or less than 220 pg/ml.

In some aspects, provided herein are methods of determining whether an individual is a candidate for angiotensin therapy by measuring prorenin levels in the patient, thereby obtaining an initial measurement of prorenin levels in the patient; and if the prorenin levels are less than 5 pg/ml, less than 10 pg/ml, less than 25 pg/ml, less than 30 pg/ml, less than 35 pg/ml, less than 40 pg/ml, less than 45 pg/ml, less than 50 pg/ml, less than 55 pg/ml, less than 60 pg/ml, less than 65 pg/ml, less than 70 pg/ml, less than 75 pg/ml, less than 80 pg/ml, less than 85 pg/ml, less than 90 pg/ml, less than 95 pg/ml, less than 100 pg/ml, less than 110 pg/ml, less than 120 pg/ml, less than 130 pg/ml, less than 140 pg/ml, less than 150 pg/ml, less than 160 pg/ml, less than 170 pg/ml, less than 180 pg/ml, less than 190 pg/ml, less than 200 pg/ml, less than 210 pg/ml, or less than 220 pg/ml, selecting the individual for angiotensin II administration.

Also provided herein are methods of treating hypotension, AKI, and/or shock in patients by measuring prorenin levels in the patient, thereby obtaining an initial measurement of prorenin levels in the patient and administering to the patient angiotensin II at an initial rate if the prorenin levels are at least 5 pg/ml, at least 10 pg/ml, at least 25 pg/ml, at least 30 pg/ml, at least 35 pg/ml, at least 40 pg/ml, at least 45 pg/ml, at least 50 pg/ml, at least 55 pg/ml, at least 60 pg/ml, at least 65 pg/ml, at least 70 pg/ml, at least 75 pg/ml, at least 80 pg/ml, at least 85 pg/ml, at least 90 pg/ml, at least 95 pg/ml, at least 100 pg/ml, at least 110 pg/ml, at least 120 pg/ml, at least 130 pg/ml, at least 140 pg/ml, at least 150 pg/ml, at least 160 pg/ml, at least 170 pg/ml, at least 180 pg/ml, at least 190 pg/ml, at least 200 pg/ml, at least 210 pg/ml, at least 220 pg/ml, at least 230 pg/ml, at least 240 pg/ml, at least 250 pg/ml, at least 260 pg/ml, at least 270 pg/ml, at least 280 pg/ml, at least 290 pg/ml, at least 300 pg/ml, at least 310 pg/ml, at least 320 pg/ml, at least 330 pg/ml, at least 340 pg/ml, 350 pg/ml, at least 360 pg/ml, at least 370 pg/ml, at least 380 pg/ml, at least 390 pg/ml, at least 400 pg/ml, at least 410 pg/ml, at least 420 pg/ml, at least 430 pg/ml, at least 440 pg/ml, or at least 450 pg/ml.

In some aspects, provided herein are methods of determining whether an individual is a candidate for angiotensin therapy by measuring prorenin levels in the patient, thereby obtaining an initial measurement of prorenin levels in the patient; and if the prorenin levels are at least 5 pg/ml, at least 10 pg/ml, at least 25 pg/ml, at least 30 pg/ml, at least 35 pg/ml, at least 40 pg/ml, at least 45 pg/ml, at least 50 pg/ml, at least 55 pg/ml, at least 60 pg/ml, at least 65 pg/ml, at least 70 pg/ml, at least 75 pg/ml, at least 80 pg/ml, at least 85 pg/ml, at least 90 pg/ml, at least 95 pg/ml, at least 100 pg/ml, at least 110 pg/ml, at least 120 pg/ml, at least 130 pg/ml, at least 140 pg/ml, at least 150 pg/ml, at least 160 pg/ml, at least 170 pg/ml, at least 180 pg/ml, at least 190 pg/ml, at least 200 pg/ml, at least 210 pg/ml, at least 220 pg/ml, at least 230 pg/ml, at least 240 pg/ml, at least 250 pg/ml, at least 260 pg/ml, at least 270 pg/ml, at least 280 pg/ml, at least 290 pg/ml, at least 300 pg/ml, at least 310 pg/ml, at least 320 pg/ml, at least 330 pg/ml, at least 340 pg/ml, 350 pg/ml, at least 360 pg/ml, at least 370 pg/ml, at least 380 pg/ml, at least 390 pg/ml, at least 400 pg/ml, at least 410 pg/ml, at least 420 pg/ml, at least 430 pg/ml, at least 440 pg/ml, or at least 450 pg/ml, selecting the individual for angiotensin II administration.

In some aspects, provided herein are methods of determining whether a patient with shock would be a candidate for angiotensin II therapy. The method may comprise measuring the a renin level in the patient, thereby obtaining an initial renin level, and if the initial renin level is at least a threshold value, the patient is a candidate for angiotensin II therapy. The method may further include administering to the patient angiotensin II at an initial rate. The threshold value may be a multiple of the value of a normal or mean renin level.

As used herein, “renin” includes plasma renin or serum renin. For example, measuring an initial renin level may include a measurement of plasma renin or serum renin. A renin level threshold may include a threshold level of plasma renin or serum renin.

The renin level threshold value may be at least 5 pg/ml, at least 10 pg/ml, at least 25 pg/ml, at least 30 pg/ml, at least 35 pg/ml, at least 40 pg/ml, at least 45 pg/ml, at least 50 pg/ml, at least 55 pg/ml, at least 60 pg/ml, at least 65 pg/ml, at least 70 pg/ml, at least 75 pg/ml, at least 80 pg/ml, at least 85 pg/ml, at least 90 pg/ml, at least 95 pg/ml, at least 100 pg/ml, at least 110 pg/ml, at least 120 pg/ml, at least 130 pg/ml, at least 140 pg/ml, at least 150 pg/ml, at least 160 pg/ml, at least 170 pg/ml, at least 180 pg/ml, at least 190 pg/ml, at least 200 pg/ml, at least 210 pg/ml, at least 220 pg/ml, at least 230 pg/ml, at least 240 pg/ml, at least 250 pg/ml, at least 260 pg/ml, at least 270 pg/ml, at least 280 pg/ml, at least 290 pg/ml, at least 300 pg/ml, at least 310 pg/ml, at least 320 pg/ml, at least 330 pg/ml, at least 340 pg/ml, 350 pg/ml, at least 360 pg/ml, at least 370 pg/ml, at least 380 pg/ml, at least 390 pg/ml, at least 400 pg/ml, at least 410 pg/ml, at least 420 pg/ml, at least 430 pg/ml, at least 440 pg/ml, or at least 450 pg/ml.

The initial PRA threshold value may be at least 0.5 ng/mL/hour, 0.6 ng/mL/hour, 0.7 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, 2.4 ng/mL/hour, 2.6 ng/mL/hour, 2.8 ng/mL/hour, 3.0 ng/mL/hour, 3.2 ng/mL/hour, 3.4 ng/mL/hour, 3.6 ng/mL/hour, 3.8 ng/mL/hour, 4.0 ng/mL/hour, 4.2 ng/mL/hour, 4.4 ng/mL/hour, 4.6 ng/mL/hour, 4.8 ng/mL/hour, 5.0 ng/mL/hour, 5.2 ng/mL/hour, 5.4 ng/mL/hour, 5.6 ng/mL/hour, 5.8 ng/mL/hour, 6.0 ng/mL/hour, 6.2 ng/mL/hour, 6.4 ng/mL/hour, 6.6 ng/mL/hour, 6.8 ng/mL/hour, 7.0 ng/mL/hour, 7.2 ng/mL/hour, 7.4 ng/mL/hour, 7.6 ng/mL/hour, 7.8 ng/mL/hour, or 8.0 ng/mL/hour.

The threshold value may depend on patient demographics and medical history. The threshold value may be determined by sample group. For example, prorenin, renin or PRA measurements from a sample group or groups with similar demographics may be obtained, and the threshold value is specified as the median or mean value of prorenin, renin, or PRA within the group. The threshold value may be a multiple of a calculated normal renin/prorenin/PRA level or a renin/prorenin/PRAlevel considered to be typical for that patient. For example, a normal or typical renin levels, prorenin levels, or PRA may be a renin level, prorenin level, or PRA that is typical for a healthy individual with the same demographics as the patient. A typical or normal PRA, prorenin level, or renin level for a male patient of a certain age may be the PRA, prorenin, or renin level for a healthy male individual of the same or similar age. A typical or normal renin level may be, for example, any value from 5 pg/ml to 55 pg/ml. A typical or normal prorenin level may be, for example, any value from 5 pg/ml to 200 pg/ml. A typical or normal PRA may be, for example, any value from 0.6 to 4.3 ng/mL/hour. The threshold level may be 2×, 3×, 4×, 5×, 6×, 7×, 8×, 9×, 10×, 11×, 12×, 13×, 14×, or 15× or more the normal or typical renin, PRA, or prorenin, level for that patient or group of patients.

The initial rate of angiotensin II may be, for example, no more than 1, 2, 3, 4, 5, 6, 7, 8, 9, 10, 11, 12, 13, 14, 15, 16, 17, 18, 19, 20, 25, 30, 35, 40, 45, or 50 ng/kg/min angiotensin II. The initial rate of angiotensin II may be, for example, at least 1, 2, 3, 4, 5, 6, 7, 8, 9, 10, 11, 12, 13, 14, 15, 16, 17, 18, 19, 20, 25, 30, 35, 40, 45, or 50 ng/kg/min angiotensin II. The initial rate may be greater than 1, 2, 3, 4, 5, 6, 7, 8, 9, 10, 11, 12, 13, 14, 15, 16, 17, 18, 19, 20, 25, 30, 35, 40, 45, or 50 ng/kg/min angiotensin II. The initial rate may be less than 1, 2, 3, 4, 5, 6, 7, 8, 9, 10, 11, 12, 13, 14, 15, 16, 17, 18, 19, 20, 25, 30, 35, 40, 45, or 50 ng/kg/min angiotensin II. The initial rate may be less than or equal to 1, 2, 3, 4, 5, 6, 7, 8, 9, 10, 11, 12, 13, 14, 15, 16, 17, 18, 19, 20, 25, 30, 35, 40, 45, or 50 ng/kg/min angiotensin II. The initial rate may be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 ng/kg/min angiotensin II. The initial rate may be about 0.1 ng/kg/min to 20 ng/kg/min, about 0.1 ng/kg/min to about 19 ng/kg/min, about 0.1 ng/kg/min to about 18 ng/kg/min, about 0.1 ng/kg/min to about 17.5 ng/kg/min, about 0.2 ng/kg/min to about 17.5 ng/kg/min, about 0.25 ng/kg/min to about 17.5 ng/kg/min, about 0.1 ng/kg/min to about 15 ng/kg/min, about 0.2 ng/kg/min to about 15 ng/kg/min, or about 0.25 ng/kg/min to about 15 ng/kg/min. The initial rate may be about 0.5 ng/kg/min to 20 ng/kg/min, about 0.5 ng/kg/min to about 19 ng/kg/min, about 0.5 ng/kg/min to about 18 ng/kg/min, about 0.5 ng/kg/min to about 17.5 ng/kg/min, about 0.75 ng/kg/min to about 17.5 ng/kg/min, about 1.0 ng/kg/min to about 17.5 ng/kg/min, about 0.5 ng/kg/min to about 15 ng/kg/min, about 0.75 ng/kg/min to about 15 ng/kg/min, or about 1.0 ng/kg/min to about 15 ng/kg/min. The initial rate may be 20 ng/kg/min to about 200 ng/kg/min, 20 ng/kg/min to about 120 ng/kg/min, 20 ng/kg/min to about 100 ng/kg/min, 20 ng/kg/min to about 90 ng/kg/min, 20 ng/kg/min to about 80 ng/kg/min, 20 ng/kg/min to about 70 ng/kg/min, 20 ng/kg/min to about 60 ng/kg/min, or 20 ng/kg/min to about 50 ng/kg/min.

Angiotensin II administration may include administering angiotensin II at an initial dose and increasing the dose prior to any additional renin, prorenin, and/or PRA measurements. For example, method may include administering angiotensin II at a rate of about 1, 2, 3, 4, 5, 6, 7, 8, 9, 10, 11, 12, 13, 14, 15, 16, 17, 18, 19, 20 ng/kg/min and increasing the rate of administration to less than or equal to 40 ng/kg/min prior to any additional PRA, prorenin, or renin measurements. The method may comprise administering angiotensin II and modulating the rate of angiotensin II prior to any subsequent PRA, prorenin, or renin measurements. For example, the method may comprise administering angiotensin II at a rate of about 10, 15, 20, 25, 30, 35, 40, 45, 50, 55, 60, 65, 70, 75, or 80 ng/kg/min and decreasing the rate of angiotensin II administration to about 1, 5, 10, 15 or 20 ng/kg/min.

The patient's PRA, prorenin, and/or renin levels may be monitored to titrate angiotensin II or an additional vasopressor. For example, the patient's renin levels can be measured by any technique known in the art, including but not limited to ELISA and immunoassays. ELISA (enzyme-linked immunosorbent assay) is a plate-based assay technique designed for detecting and quantifying substances (e.g., renin). In an ELISA, an antigen must be immobilized on a solid surface and then complexed with an antibody (e.g., an antibody specific for renin) that is linked to an enzyme. Detection may be accomplished by assessing the conjugated enzyme activity via incubation with a substrate to produce a measureable product. The most crucial element of the detection strategy is a highly specific antibody-antigen interaction.

In some embodiments, PRA, prorenin, and/or renin levels are measured prior to administering angiotensin II or a composition comprising angiotensin II, the composition is administered, and, after a subsequent period of time, an additional PRA, prorenin, and/or renin level is measured.

The methods provided herein include modulating the dose of angiotensin II in response to renin level measurements, prorenin level measurement, or PRA measurements. The rate of administration of angiotensin II may be any rate disclosed herein. The method may further comprise measuring the patient's PRA, prorenin, and/or renin levels after a period of time.

In some embodiments, the method further comprises decreasing or maintaining the rate of administration of angiotensin II if the patient's measured PRA or measured renin levels are less than a threshold value. In some embodiments, the method further comprises measuring the patient's PRA or renin levels after a period of time, and if the patient's PRA or measured renin levels are more than a threshold value, increasing or maintaining the rate of administration of angiotensin II. The renin threshold value may be at least 25 pg/ml, at least 30 pg/ml, at least 35 pg/ml, at least 40 pg/ml, at least 45 pg/ml, at least 50 pg/ml, at least 55 pg/ml, at least 60 pg/ml, at least 65 pg/ml, at least 70 pg/ml, at least 75 pg/ml, at least 80 pg/ml, at least 85 pg/ml, at least 90 pg/ml, at least 95 pg/ml, at least 100 pg/ml, at least 110 pg/ml, at least 120 pg/ml, at least 130 pg/ml, at least 140 pg/ml, at least 150 pg/ml, at least 160 pg/ml, at least 170 pg/ml, at least 180 pg/ml, at least 190 pg/ml, at least 200 pg/ml, at least 210 pg/ml, at least 220 pg/ml, at least 230 pg/ml, at least 240 pg/ml, at least 250 pg/ml, at least 260 pg/ml, at least 270 pg/ml, at least 280 pg/ml, at least 290 pg/ml, at least 300 pg/ml, at least 310 pg/ml, at least 320 pg/ml, at least 330 pg/ml, at least 340 pg/ml, 350 pg/ml, at least 360 pg/ml, at least 370 pg/ml, at least 380 pg/ml, at least 390 pg/ml, at least 400 pg/ml, at least 410 pg/ml, at least 420 pg/ml, at least 430 pg/ml, at least 440 pg/ml, or at least 450 pg/ml. The threshold value may depend on patient demographics and medical history.

The initial PRA threshold value may be at least 0.5 ng/mL/hour, 0.6 ng/mL/hour, 0.7 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, 2.4 ng/mL/hour, 2.6 ng/mL/hour, 2.8 ng/mL/hour, 3.0 ng/mL/hour, 3.2 ng/mL/hour, 3.4 ng/mL/hour, 3.6 ng/mL/hour, 3.8 ng/mL/hour, 4.0 ng/mL/hour, 4.2 ng/mL/hour, 4.4 ng/mL/hour, 4.6 ng/mL/hour, 4.8 ng/mL/hour, 5.0 ng/mL/hour, 5.2 ng/mL/hour, 5.4 ng/mL/hour, 5.6 ng/mL/hour, 5.8 ng/mL/hour, 6.0 ng/mL/hour, 6.2 ng/mL/hour, 6.4 ng/mL/hour, 6.6 ng/mL/hour, 6.8 ng/mL/hour, 7.0 ng/mL/hour, 7.2 ng/mL/hour, 7.4 ng/mL/hour, 7.6 ng/mL/hour, 7.8 ng/mL/hour, or 8.0 ng/mL/hour.

The period of time may be, for example, about 5 minutes, about 15 minutes, about 30 minutes, about 1 hour, about 2 hours, about 3, hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 24 hours, about 30 hours, about 36 hours, about 48 hours, or about 72 hours. The period of time may be about 1 day, about 2 days, about 3 days, about 4 days, about 5 days, about 6 days, or about 7 days.

The method disclosed herein may require measuring the patient's renin levels at least once, at least twice, at least 5, at least 10, at least 15 at least 20, at least 25, at least 50 or at least 100 times over the course of angiotensin II administration.

In some embodiments, the angiotensin II is administered until renin levels are lowered to a normal or a typical renin level for that patient. Angiotensin II may be administered for about 15 minutes, about 30 minutes, about 1 hour, about 2 hours, about 3, hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 24 hours, about 30 hours, about 36 hours, about 48 hours, or about 72 hours. Angiotensin II may be administered for about 1 day, about 2 days, about 3 days, about 4 days, about 5 days, about 6 days, about 7 days, about two weeks, about one month, or about three months. Angiotensin II may be administered for about 1 hour to about 7 days, about 1 hour to about 72 hours, about 1 hour to about 48 hours, about 1 hour to about 24 hours, about 1 hour to about 12 hours, or about 1 day to about 7 days.

In some embodiments, the angiotensin II is administered until renin levels are lowered to below specific value (e.g., less than 500 pg/ml, less than 450 pg/ml, less than 400 pg/ml, less than 350 pg/ml, less than 300 pg/ml, less than 250 pg/ml, less than 200 pg/ml, less than 190 pg/ml, less than 180 pg/ml, less than 170 pg/ml, less than 160 pg/ml, less than 150 pg/ml, less than 140 pg/ml, less than 130 pg/ml, less than 120 pg/ml, less than 110 pg/ml, less than 100 pg/ml, less than 90 pg/ml, less than 80 pg/ml, less than 70 pg/ml, less than 60 pg/ml, less than 50 pg/ml, less than 40 pg/ml, less than 30 pg/ml, or less than 20 pg/ml.)

In some embodiments, the angiotensin II is administered until PRA levels are lowered to below specific value (e.g., less than 0.5 ng/mL/hour, 0.6 ng/mL/hour, 0.7 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, 2.4 ng/mL/hour, 2.6 ng/mL/hour, 2.8 ng/mL/hour, 3.0 ng/mL/hour, 3.1 ng/mL/hour, 3.2 ng/mL/hour, 3.4 ng/mL/hour, 3.6 ng/mL/hour, 3.8 ng/mL/hour, 4.0 ng/mL/hour, 4.2 ng/mL/hour, 4.3 ng/mL/hour, 4.4 ng/mL/hour, 4.8 ng/mL/hour, 5.0 ng/mL/hour, 5.2 ng/mL/hour, 5.4 ng/mL/hour, 5.6 ng/mL/hour, 5.8 ng/mL/hour, 6.0 ng/mL/hour, 6.2 ng/mL/hour, 6.4 ng/mL/hour, 6.6 ng/mL/hour, 6.8 ng/mL/hour or 7.0 ng/mL/hour)

Angiotensin II may be particularly useful for patients who require potentially harmful doses of vasopressors. Thus, in some embodiments, the invention relates to methods of treating hypotension, wherein, prior to administering the composition, the patient is receiving dopamine, dobutamine, norepinephrine, epinephrine, phenylephrine, terlipressin, vasopressin, a vasopressin analogue, or midodrine as a vasopressor. The vasopressor may be, for example, a catecholamine. The term “catecholamine”, as used herein, refers to dopamine, norepinephrine, phenylephrine, and epinephrine and their prodrugs, structural analogs, or derivatives that induce similar physiological effects in humans, e.g., raise mean arterial pressure in healthy human subjects. In certain embodiments, the catecholamine may be dopamine, norepinephrine, or epinephrine. The vasopressor may be vasopressin or a vasopressin analogue. A vasopressin analogue may be, for example, terlipressin, argipressin, desmopressin, felypressin, lypressin, or ornipressin. In some embodiments, a method comprises administering two or more of angiotensin II, a catecholamine, vasopressin, a vasopressin analog, dobutamine, and midodrine to a patient. For example, a method may comprise administering angiotensin II, a catecholamine, and either vasopressin or a vasopressin analog to a patient. A method may comprise administering angiotensin II, a catecholamine, and vasopressin to a patient.

In certain embodiments, if the measured renin levels fall below a certain threshold after angiotensin administration (e.g., less than 250 pg/ml, less than 200 pg/ml, less than 150 pg/ml, less than 100 pg/ml, less than 50 pg/ml, less than 25 pg/ml, or less than 10 pg/ml), then the rate at which a vasopressor or angiotensin II is administered is maintained or decreased. Alternatively, in certain embodiments, if the measured renin levels do not fall below a certain threshold after angiotensin administration (e.g., less than 250 pg/ml, less than 200 pg/ml, less than 150 pg/ml, less than 100 pg/ml, or less than 50 pg/ml), then the rate at which a vasopressor or angiotensin II is administered is maintained or increased.

In certain embodiments, if the measured PRA falls below a certain threshold after angiotensin administration (e.g., less than 3.0 ng/mL/hour, 3.1 ng/mL/hour, 3.2 ng/mL/hour, 3.4 ng/mL/hour, 3.6 ng/mL/hour, 3.8 ng/mL/hour, 4.0 ng/mL/hour, 4.2 ng/mL/hour, 4.3 ng/mL/hour, 4.4 ng/mL/hour, 4.8 ng/mL/hour, 5.0 ng/mL/hour, 5.2 ng/mL/hour, 5.4 ng/mL/hour, 5.6 ng/mL/hour, 5.8 ng/mL/hour, 6.0 ng/mL/hour, 6.2 ng/mL/hour, 6.4 ng/mL/hour, 6.6 ng/mL/hour, 6.8 ng/mL/hour or 7.0 ng/mL/hour), then the rate at which a vasopressor or angiotensin II is administered is maintained or decreased. Alternatively, in certain embodiments, if the measured renin levels do not fall below a certain threshold after angiotensin administration (e.g., less than 3.0 ng/mL/hour, 3.1 ng/mL/hour, 3.2 ng/mL/hour, 3.4 ng/mL/hour, 3.6 ng/mL/hour, 3.8 ng/mL/hour, 4.0 ng/mL/hour, 4.2 ng/mL/hour, 4.3 ng/mL/hour, 4.4 ng/mL/hour, 4.8 ng/mL/hour, 5.0 ng/mL/hour, 5.2 ng/mL/hour, 5.4 ng/mL/hour, 5.6 ng/mL/hour, 5.8 ng/mL/hour, 6.0 ng/mL/hour, 6.2 ng/mL/hour, 6.4 ng/mL/hour, 6.6 ng/mL/hour, 6.8 ng/mL/hour or 7.0 ng/mL/hour), then the rate at which a vasopressor or angiotensin II is administered is maintained or increased.

In some embodiments, the rate at which a vasopressor is administered may be decreased after each renin measurement (i.e., any vasopressor, including angiotensin II, a catecholamine, vasopressin, etc.) depending on whether the difference between the initial renin level and the measured renin level is more than a target value. For example, in some embodiments, if the difference between the initial renin level and the measured renin level is at least 1, 5, 10, 25, 50, 75, 100, 150, 200, 250, 300, 350, 400, or 500 pg/ml, then the rate at which a vasopressor is administered to the patient is decreased or maintained (i.e., any vasopressor, including angiotensin II, a catecholamine, vasopressin, etc.).

In some embodiments, the rate at which a vasopressor is administered may be decreased after each PRA measurement (i.e., any vasopressor, including angiotensin II, a catecholamine, vasopressin, etc.) depending on whether the difference between the initial PRA level and the measured PRA level is more than a target value. For example, in some embodiments, if the difference between the initial PRA level and the measured PRA level is at least 0.05 ng/mL/hour, 0.1 ng/mL/hour, 0.5 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, 2.4 ng/mL/hour), then the rate at which a vasopressor is administered to the patient is decreased or maintained (i.e., any vasopressor, including angiotensin II, a catecholamine, vasopressin, etc.).

In some embodiments, the rate at which a vasopressor is administered may be increased after a renin measurement (i.e., any vasopressor, including angiotensin II, a catecholamine, vasopressin, etc.) depending on whether the difference between the initial renin level and the measured renin level is less than a target value. For example, in some embodiments, if the difference between the initial renin level and the measured renin level is less than 1, 5, 10, 25, 50, 75, 100, 150, 200, 250, 300, 350, 400, or 500 pg/ml, then the rate at which a vasopressor is administered to the patient is increased or maintained (i.e., any vasopressor, including angiotensin II, a catecholamine, vasopressin, etc.).

In some embodiments, the rate at which a vasopressor is administered may be increased after a PRA measurement (i.e., any vasopressor, including angiotensin II, a catecholamine, vasopressin, etc.) depending on whether the difference between the initial PRA level and the measured PRA level is less than a target value. For example, in some embodiments, if the difference between the initial PRA level and the measured PRA level is less than 0.05 ng/mL/hour, 0.1 ng/mL/hour, 0.5 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, or 2.4 ng/mL/hour, then the rate at which a vasopressor is administered to the patient is increased or maintained (i.e., any vasopressor, including angiotensin II, a catecholamine, vasopressin, etc.).

In some embodiments, if the measured renin level is at least 5, 10, 25, 50, 75, 100, 150, 200, 250, 300, 350, 400, or 500 pg/ml lower than the initial renin level, then the rate at which a vasopressor is administered to the patient is decreased (i.e., any vasopressor, including angiotensin II, a catecholamine, vasopressin, etc.). Alternatively, in certain embodiments, if the measured renin level is about the same or higher after angiotensin II administration, then the rate of angiotensin II administration will be increased.

In some embodiments, if the measured PRA is lower than the initial PRA level, then the rate at which a vasopressor is administered to the patient is decreased (i.e., any vasopressor, including angiotensin II, a catecholamine, vasopressin, etc.). Alternatively, in certain embodiments, if the measured PRA level is about the same or higher after angiotensin II administration, then the rate of angiotensin II administration will be increased.

The method may comprise measuring a mean arterial pressure (MAP) of the patient (in addition to measuring renin levels) a subsequent period of time after administering to the patient angiotensin II. The term “mean arterial pressure” or “MAP” refers to the average arterial pressure during a single cardiac cycle. The method may comprise decreasing the rate at which a vasopressor and/or angiotensin II is administered, if the measured mean arterial pressure is at or above 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 mm Hg (referred to as a target value or target MAP herein). For example, the method may comprise decreasing the rate at which a vasopressor and/or angiotensin II is administered if the measured mean arterial pressure is at or above 75 mm Hg. The method may further comprise increasing the rate at which the angiotensin II is administered if the measured mean arterial pressure is less than 75 mm Hg (or less than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 mm Hg, e.g., wherein the target MAP corresponds to the target MAP for decreasing the rate at which the vasopressor is administered).

A patient may have a known initial mean arterial pressure prior to administering the angiotensin II (referred to as an initial mean arterial pressure herein, which is distinguished from a measured mean arterial pressure that is obtained after administering the composition to the patient). For example, the method may comprise measuring a mean arterial pressure and renin levels prior to administering angiotensin II to the patient. The method may comprise decreasing the rate at which a vasopressor or angiotensin II is administered if the measured mean arterial pressure (obtained a subsequent period of time after administering to the patient the composition) is at least 1, 2, 3, 4, 5, 6, 7, 8, 9, 10, 11, 12, 13, 14, 15, 16, 17, 18, 19, or 20 mm Hg higher than the initial mean arterial pressure.

In some embodiments, the patient is receiving at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μg/kg/min of norepinephrine prior to administration of angiotensin II. For example, prior to administering the composition, the patient may be receiving at least 0.1 μg/kg/min of norepinephrine. In some embodiments, the patient may be receiving at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/min of norepinephrine prior to administering the composition.

Alternatively, hypotension may be treated with epinephrine. Thus, in some embodiments, the patient may be receiving at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μg/kg/min of epinephrine prior to initiation of angiotensin II therapy. For example, prior to administering the composition, the patient may be receiving at least 0.1 μg/kg/min of epinephrine. In some embodiments, the patient may be receiving at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/min of epinephrine prior to initiation of angiotensin II therapy.

Alternatively, hypotension may be treated with dopamine. Thus, in some embodiments, the patient may be receiving at least 5, 6, 7, 8, 9, 10, 11, 12, 13, 14, 15, 16, 17, 18, 19, 20, 21, 22, 23, 24, or 25 μg/kg/min of dopamine prior to initiation of angiotensin II therapy. For example, prior to administering the composition, the patient may be receiving at least 5 μg/kg/min of dopamine. In some embodiments, the patient may be receiving at least 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, or 2500 μg/min of dopamine prior to initiation of angiotensin II therapy.

Alternatively, hypotension may be treated with vasopressin. Thus, in some embodiments, the patient may be receiving at least 1, 2, 3, 4, 5, 6, 7, 8, 9, 10, 11, 12, 13, 14, 15, 16, 17, 18, 19, 20, 21, 22, 23, 24, or 25 mU/kg/min vasopressin. In some embodiments, the patient may be receiving at least 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.25, 0.30, 0.40, 0.50, 0.60, 0.70, 0.80, 0.90, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.0 U/min vasopressin. For example, prior to administering the composition, the patient may be receiving at least 0.01 U/min of vasopressin.

In some embodiments, the invention relates to methods of treating hypotension wherein the patient has a cardiovascular sequential organ failure assessment score (“SOFA score”) of 1 or greater prior to initiation of angiotensin II therapy. For example, a patient may have a cardiovascular SOFA score of 1, 2, 3, or 4. In some embodiments, the patient has a cardiovascular SOFA score of 2, 3, or 4. In other embodiments, the patient has a cardiovascular SOFA score of 3 or 4. In some embodiments, the patient has a cardiovascular SOFA score of 4 prior to initiation of angiotensin II therapy.

The patient's mean arterial pressure may be monitored to titrate angiotensin II or the vasopressor. For example, the patient's mean arterial pressure may be monitored with an indwelling arterial line or by other suitable methods. In some embodiments, an initial mean arterial pressure is measured prior to administering angiotensin II or a composition comprising angiotensin II, the composition is administered, and, after a period of time, an additional mean arterial pressure is measured. The period of time may be, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 135, 150, 165, 180, 195, 210, 225, or 240 minutes, or about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24, 25, 26, 27, 28, 29, 30, 31, 32, 33, 34, 35, 36, 37, 38, 39, 40, 41, 42, 43, 44, 45, 46, 47, or 48 hours or longer. Preferably, the period of time is less than two hours, most preferably about one hour or less.

In certain embodiments, if the measured mean arterial pressure meets or exceeds a target value and/or if the patient's renin levels fall below a target value (e.g., less than 250 pg/ml, less than 200 pg/ml, less than 150 pg/ml, less than 100 pg/ml, or less than 50 pg/ml), then the rate at which a vasopressor or angiotensin II is administered is decreased. The target value for MAP may be, for example, 60, 61, 62, 63, 64, 65, 66, 67, 68, 69, 70, 71, 72, 73, 74, 75, 76, 77, 78, 79, or 80 mm Hg. In certain preferred embodiments, if the measured mean arterial pressure is at or above 75 mm Hg, then the rate at which a vasopressor and/or angiotensin II is administered is decreased (i.e., any vasopressor, including angiotensin II, a catecholamine, vasopressin, etc.). In some embodiments, if the patient's renin level fall below a 150 pg/ml, then the rate at which a vasopressor angiotensin II may be decreased.

In certain embodiments, if the patient's PRA levels fall below a target value (e.g., less than 0.05 ng/mL/hour, 0.1 ng/mL/hour, 0.5 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, 2.4 ng/mL/hour), then the rate at which a vasopressor or angiotensin II is administered is decreased.

In some embodiments, the rate of administration at which a vasopressor is administered is decreased (i.e., any vasopressor, including angiotensin II, a catecholamine, vasopressin, etc.) in response to additional measurements of renin and/or PRA levels.

In some embodiments, if the difference between the initial renin level and the measured renin level meets or exceeds a target value, then the rate at which a vasopressor is administered is maintained or decreased (i.e., any vasopressor, including angiotensin II, a catecholamine, vasopressin, etc.). The target value may be, for example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140 145, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 pg/ml.

In some embodiments, if the difference between the initial PRA and the measured PRA meets or exceeds a target value, then the rate at which a vasopressor is administered is maintained or decreased (i.e., any vasopressor, including angiotensin II, a catecholamine, vasopressin, etc.). The target value may be, for example, at least 0.05 ng/mL/hour, 0.06 ng/mL/hour, 0.07 ng/mL/hour, 0.08 ng/mL/hour, 0.09 ng/mL/hour, 0.1 ng/mL/hour, 0.2 ng/mL/hour, 0.3 ng/mL/hour, 0.4 ng/mL/hour, 0.5 ng/mL/hour, 0.6 ng/mL/hour, 0.7 ng/mL/hour, 0.8 ng/mL/hour, 0.9 ng/mL/hour, 1.0 ng/mL/hour, 1.2 ng/mL/hour, 1.3 ng/mL/hour, 1.4 ng/mL/hour, 1.5 ng/mL/hour, 1.7 ng/mL/hour, 1.8 ng/mL/hour, or 2.0 ng/mL/hour.

In some embodiments, if the measured renin level is at least 5, 10, 25, 50, 75, 100, 150, 200, 250, 300, 350, 400, or 500 pg/ml lower than the initial renin level, then the rate at which a vasopressor is administered to the patient is decreased (i.e., any vasopressor, including angiotensin II, a catecholamine, vasopressin, etc.).

In some embodiments, if the measured PRA is at least 0.05, 0.1, 0.2, 0.3, 0.5, 0.7, 0.9, 1.0, 1.5 or 2.0 ng/mL/hour lower than the initial PRA, then the rate at which a vasopressor is administered to the patient is decreased (i.e., any vasopressor, including angiotensin II, a catecholamine, vasopressin, etc.).

In some embodiments, the rate of administration at which a vasopressor is administered is increased (i.e., any vasopressor, including angiotensin II, a catecholamine, vasopressin, etc.) in response to additional measurements of renin and/or PRA levels.

In some embodiments, if the difference between the initial renin level and the measured renin level is less than a target value, then the rate at which a vasopressor is administered is increased or maintained (i.e., any vasopressor, including angiotensin II, a catecholamine, vasopressin, etc.). The target value may be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140 145, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 pg/ml.

In some embodiments, if the difference between the initial PRA and the measured PRA is less than a target value, then the rate at which a vasopressor is administered is increased or maintained (i.e., any vasopressor, including angiotensin II, a catecholamine, vasopressin, etc.). The target value may be, for example, 0.05 ng/mL/hour, 0.06 ng/mL/hour, 0.07 ng/mL/hour, 0.08 ng/mL/hour, 0.09 ng/mL/hour, 0.1 ng/mL/hour, 0.2 ng/mL/hour, 0.3 ng/mL/hour, 0.4 ng/mL/hour, 0.5 ng/mL/hour, 0.6 ng/mL/hour, 0.7 ng/mL/hour, 0.8 ng/mL/hour, 0.9 ng/mL/hour, 1.0 ng/mL/hour, 1.2 ng/mL/hour, 1.3 ng/mL/hour, 1.4 ng/mL/hour, 1.5 ng/mL/hour, 1.7 ng/mL/hour, 1.8 ng/mL/hour, or 2.0 ng/mL/hour.

In some embodiments, if the measured renin level is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140 145, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 pg/ml lower than the initial renin level, then the rate at which a vasopressor is administered to the patient is increased or maintained (i.e., any vasopressor, including angiotensin II, a catecholamine, vasopressin, etc.).

In some embodiments, if the measured PRA is less than 0.05 ng/mL/hour, 0.06 ng/mL/hour, 0.07 ng/mL/hour, 0.08 ng/mL/hour, 0.09 ng/mL/hour, 0.1 ng/mL/hour, 0.2 ng/mL/hour, 0.3 ng/mL/hour, 0.4 ng/mL/hour, 0.5 ng/mL/hour, 0.6 ng/mL/hour, 0.7 ng/mL/hour, 0.8 ng/mL/hour, 0.9 ng/mL/hour, 1.0 ng/mL/hour, 1.2 ng/mL/hour, 1.3 ng/mL/hour, 1.4 ng/mL/hour, 1.5 ng/mL/hour, 1.7 ng/mL/hour, 1.8 ng/mL/hour, 2.0 ng/mL/hour, 2.2 ng/mL/hour, 2.3 ng/mL/hour, 2.4 ng/mL/hour, 2.5 ng/mL/hour, 2.7 ng/mL/hour, 2.8 ng/mL/hour, or 3.0 ng/mL/hour lower than the initial PRA, then the rate at which a vasopressor is administered to the patient is increased or maintained (i.e., any vasopressor, including angiotensin II, a catecholamine, vasopressin, etc.).

In some embodiments, if the measured renin level is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140 145, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 pg/ml lower than the initial renin level, then the rate at which a vasopressor is administered to the patient is decreased or maintained (i.e., any vasopressor, including angiotensin II, a catecholamine, vasopressin, etc.).

In some embodiments, if the measured PRA is at least 0.05 ng/mL/hour, 0.06 ng/mL/hour, 0.07 ng/mL/hour, 0.08 ng/mL/hour, 0.09 ng/mL/hour, 0.1 ng/mL/hour, 0.2 ng/mL/hour, 0.3 ng/mL/hour, 0.4 ng/mL/hour, 0.5 ng/mL/hour, 0.6 ng/mL/hour, 0.7 ng/mL/hour, 0.8 ng/mL/hour, 0.9 ng/mL/hour, 1.0 ng/mL/hour, 1.2 ng/mL/hour, 1.3 ng/mL/hour, 1.4 ng/mL/hour, 1.5 ng/mL/hour, 1.7 ng/mL/hour, 1.8 ng/mL/hour, 2.0 ng/mL/hour, 2.2 ng/mL/hour, 2.3 ng/mL/hour, 2.4 ng/mL/hour, 2.5 ng/mL/hour, 2.7 ng/mL/hour, 2.8 ng/mL/hour, or 3.0 ng/mL/hour lower than the initial PRA, then the rate at which a vasopressor is administered to the patient is decreased or maintained (i.e., any vasopressor, including angiotensin II, a catecholamine, vasopressin, etc.).

In some embodiments, the rate of administration at which a vasopressor is administered is decreased (i.e., any vasopressor, including angiotensin II, a catecholamine, vasopressin, etc.) in response to MAP levels. In some embodiments, if the difference between the measured mean arterial pressure and the initial mean arterial pressure meets or exceeds a target value, then the rate at which a vasopressor is administered is decreased (i.e., any vasopressor, including angiotensin II, a catecholamine, vasopressin, etc.). The target value may be, for example, 1, 2, 3, 4, 5, 6, 7, 8, 9, 10, 11, 12, 13, 14, 15, 16, 17, 18, 19, 20, 21, 22, 23, 24, or 25 mm Hg.

In some embodiments, if the measured renin level is at least 5, 10, 25, 50, 75, 100, 150, 200, 250, 300, 350, 400, or 500 pg/ml lower than the initial renin level, then the rate at which a vasopressor is administered to the patient is decreased (i.e., any vasopressor, including angiotensin II, a catecholamine, vasopressin, etc.). In certain embodiments, if the measured mean arterial pressure is at least 10 mm Hg higher than the initial mean arterial pressure, then the rate at which a vasopressor is administered is decreased (i.e., any vasopressor, including angiotensin II).

The mean arterial pressure may be measured more than once; for example, the mean arterial pressure may be measured 1, 2, 3, 4, 5, 6, 7, 8, 9, 10 or more times, or even continuously or substantially continuously. The rate at which a vasopressor is administered may be decreased in response to each measurement of MAP (i.e., any vasopressor, including angiotensin II, a catecholamine, vasopressin, etc.), depending on whether the mean arterial pressure meets or exceeds a target value. Similarly, the rate at which a vasopressor is administered may be increased after a MAP measurement (i.e., any vasopressor, including angiotensin II, a catecholamine, vasopressin, etc., if the measured mean arterial pressure is less than a target value. The target value for MAP may be, for example, 60, 61, 62, 63, 64, 65, 66, 67, 68, 69, 70, 71, 72, 73, 74, 75, 76, 77, 78, 79, or 80 mm Hg.

Renin or prorenin levels may be measured more than once; for example, the renin level may be measured 1, 2, 3, 4, 5, 6, 7, 8, 9, 10 or more times, or even continuously or substantially continuously. The rate at which a vasopressor is administered may be decreased in response to each measurement of renin (i.e., any vasopressor, including angiotensin II, a catecholamine, vasopressin, etc.), depending on whether the renin level meets or is lower than a target value. Similarly, the rate at which a vasopressor is administered may be increased after a renin measurement (i.e., any vasopressor, including angiotensin II, a catecholamine, vasopressin, etc.), if the renin level is more than a target value. The target value may be 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140 145, 150, 160, 170, 180, 190, 200, 210, 220, 230, 240, 250, 260, 270, 280, 290, 300, 310, 320, 330, 340, 350, 360, 370, 380, 390, or 400 pg/ml.

PRA may be measured more than once; for example, PRA may be measured 1, 2, 3, 4, 5, 6, 7, 8, 9, 10 or more times, or even continuously or substantially continuously. The rate at which a vasopressor is administered may be decreased in response to each measurement of PRA (i.e., any vasopressor, including angiotensin II, a catecholamine, vasopressin, etc.), depending on whether the PRA meets or is lower than a target value. Similarly, the rate at which a vasopressor is administered may be increased after a PRA measurement (i.e., any vasopressor, including angiotensin II, a catecholamine, vasopressin, etc.), if the PRA level is more than a target value. The target value may be 0.5 ng/mL/hour, 0.6 ng/mL/hour, 0.7 ng/mL/hour, 1.0 ng/mL/hour, 1.2 ng/mL/hour, 1.4 ng/mL/hour, 1.6 ng/mL/hour, 1.8 ng/mL/hour, 2.0 ng/mL/hour, 2.2 ng/mL/hour, 2.4 ng/mL/hour, 2.6 ng/mL/hour, 2.8 ng/mL/hour, 3.0 ng/mL/hour, 3.1 ng/mL/hour, 3.2 ng/mL/hour, 3.4 ng/mL/hour, 3.6 ng/mL/hour, 3.8 ng/mL/hour, 4.0 ng/mL/hour, 4.2 ng/mL/hour, 4.3 ng/mL/hour, 4.4 ng/mL/hour, 4.8 ng/mL/hour, 5.0 ng/mL/hour.

In some embodiments, if the patient's measured mean arterial pressure is at or above 75 mm Hg and/or if the patient's renin levels and/or PRA are below a target value (e.g., a blood plasma renin threshold level disclosed herein), then the rate at which a vasopressor is administered to the patient is decreased (i.e., any vasopressor, including angiotensin II). In some embodiments, if the patient's measured mean arterial pressure is at or above 55, 56, 57, 58, 59 60, 61, 62, 62 64, 65, 66, 67, 68, 69, 70, 71, 72, 73, 74, 75, 76, 77, 78, 79, 80, 81, 82, 83, 84, or 85 mm Hg, then the rate at which a vasopressor is administered to the patient is decreased (i.e., any vasopressor, including angiotensin II, a catecholamine, vasopressin, etc.). In some embodiments, if the measured mean arterial pressure is at least 10 mm Hg higher than the initial mean arterial pressure, then the rate at which a vasopressor is administered to the patient is decreased (i.e., any vasopressor, including angiotensin II, a catecholamine, vasopressin, etc.). In some embodiments, if the measured MAP is at least 1, 2, 3, 4, 5, 6, 7, 8, 9, 10, 11, 12, 13, 14, 15, 16, 17, 18, 19, 20, 21, 22, 23, 24, or 25 mm Hg higher than the initial mean arterial pressure, then the rate at which a vasopressor is administered to the patient is decreased (i.e., any vasopressor, including angiotensin II, a catecholamine, vasopressin, etc.).

Similarly, the rate at which a vasopressor is administered may be increased after a measurement (i.e., any vasopressor, including angiotensin II, a catecholamine, vasopressin, etc.), if the difference between the measured mean arterial pressure and the initial mean arterial pressure is less than a target value. The target value may be, for example, 1, 2, 3, 4, 5, 6, 7, 8, 9, 10, 11, 12, 13, 14, 15, 16, 17, 18, 19, 20, 21, 22, 23, 24, or 25 mm Hg.

In certain embodiments, the rate at which a vasopressor is administered is decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more (i.e., a vasopressor other than angiotensin II, e.g., which the patient is receiving prior to administering the composition). Thus, for example, the rate at which norepinephrine is administered may be decreased by at least 15%. In other embodiments, the rate at which a vasopressor is administered may be decreased by at least 60%. In some embodiments, the rate at which a vasopressor (other than angiotensin II) is administered is decreased to 0 μg/kg/min (or μg/min, 0 U/kg/min, or 0 U/min).

A vasopressor may be titrated down while monitoring a patient's MAP, the patient's renin levels, the patient's prorenin levels, and/or the patient's PRA, and titration may occur over the course of minutes to hours. Thus, the rate at which a vasopressor is administered may be decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more over the course of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 135, 150, 165, 180, 195, 210, 225, or 240 minutes, or over the course of about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24, 25, 26, 27, 28, 29, 30, 31, 32, 33, 34, 35, 36, 37, 38, 39, 40, 41, 42, 43, 44, 45, 46, 47, or 48 hours or longer.

The rate of administration may be titrated by administering angiotensin II at an initial rate and then increasing or decreasing the rate of administration. In some cases, the patient may be administered an initial bolus of angiotensin II followed by the administration of angiotensin II at a lower rate. Alternatively, the patient may be administered angiotensin II at a low rate followed by gradual, elevated rates. Thus, in some embodiments, the method further comprises increasing the rate at which angiotensin II is administered, and in other embodiments, the method further comprises decreasing the rate at which angiotensin II is administered. For example, angiotensin II may be administered at an initial rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min, and the rate may be increased to a final rate of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min. Alternatively, angiotensin II may be administered at an initial rate of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/kg/min, and the rate may be decreased to a final rate of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20 ng/kg/min. Angiotensin II may be titrated while monitoring a patient's MAP, the patient's renin levels, the patient's prorenin levels, and/or the patient's PRA, and titration may occur over the course of minutes to hours. Thus, the rate at which at which angiotensin II is administered may be increased or decreased over the course of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 135, 150, 165, 180, 195, 210, 225, or 240 minutes, or over the course of about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24, 25, 26, 27, 28, 29, 30, 31, 32, 33, 34, 35, 36, 37, 38, 39, 40, 41, 42, 43, 44, 45, 46, 47, 48, 72, hours or longer, or over the course of one day, two days, three days, four days, five days, six days, or seven days or longer.

Angiotensin II may be administered as long as necessary to maintain a MAP above a target value, suppress renin levels below a certain value, suppress PRA below a certain value, or keep prorenin levels within a normal range. Alternatively, angiotensin II may be administered until the patient's MAP, renin levels, prorenin levels, and/or PRA can be maintained at a lower dose of a vasopressor other than angiotensin II. In some embodiments, the composition is administered until the mean arterial pressure of the patient can be maintained at or above 70 mm Hg with less than 0.1 μg/kg/min norepinephrine, less than 0.1 μg/kg/min epinephrine, less than 15 μg/kg/min dopamine, or less than 0.01 U/min vasopressin.

The methods disclosed herein can use any suitable form or analog of angiotensin II that exhibits the desired effect of increasing MAP, decreasing renin levels, keeping prorenin levels within a normal range, and/or decreasing the patient's PRA in human subjects. In some embodiments, the angiotensin II has the sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. Preferably, the angiotensin II has the sequence set forth in SEQ ID NO:1.

In some embodiments, the angiotensin II is selected from 5-L-valine angiotensin II; 1-L-asparagine-5-L-valine angiotensin II; 1-L-asparagine-5-L-isoleucine angiotensin II; or 1-L-asparagine-5-L-isoleucine angiotensin II, preferably 5-L-isoleucine angiotensin II. The angiotensin II may be formulated as a pharmaceutically acceptable salt, for example, as an acetate salt.

Angiotensin therapy can include administration of angiotensin II in the form of a composition comprising angiotensin II. The composition may be formulated with varying concentrations of angiotensin II. Thus, in certain embodiments, the composition comprises angiotensin II at a concentration of about 1, 2, 3, 4, 5, 6, 7, 8, 9, 10, 15, 20, 25, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 μg/ml. In other embodiments, the composition comprises angiotensin II at a concentration of about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, or 25.0 mg/ml. Thus, in certain embodiments, the composition comprises angiotensin II at a concentration of about 2.5 mg/mL.

In certain embodiments, the composition comprises an excipient, such as mannitol.

In certain embodiments, the composition is suitable for parenteral administration, such as injection or intravenous infusion, preferably intravenous infusion.

In some embodiments, the patient has sepsis. The patient may have septic shock, distributive shock, or cardiogenic shock. In some embodiments, the patient has acute respiratory distress syndrome (ARDS), acute kidney injury, hepatorenal syndrome, and/or cirrhosis.

In some embodiments, the patient is a mammal, such as a primate, ovine, porcine, canine, or rodent, preferably a human.

The rate of administration of the angiotensin II can be modulated manually and/or automatically in response to measurements of the patient's renin levels, prorenin levels, and/or PRA obtained periodically or sporadically during treatment, e.g., to maintain a predetermined renin level, prorenin level, or PRA.

The rate of administration of the angiotensin II can be modulated manually and/or automatically in response to measurements of the patient's mean arterial pressure obtained periodically or sporadically during treatment, e.g., to maintain a mean arterial pressure at this level, or within a predetermined range (e.g., 80-110 mm Hg).

Those of skill in the art will recognize that in the context of the present invention, anti-hypotensive therapeutics can be administered in any suitable way, but are typically administered by continuous infusion. Accordingly, increasing or decreasing a rate of administration can be accomplished by changing the rate of flow of an intravenous drip, changing the concentration of the agent in an intravenous drip, etc. However, the manner in which the rate of administration is changed will depend on the mode of administration of the therapeutic. Where the therapeutic is administered transmucosally or transdermally, the rate may be increased by changing to a higher-release-rate patch or transdermal composition for example. Where the therapeutic is administered orally, the rate may be increased by switching to a higher-dose form, administering additional doses, or administering controlled-release dosage forms with a higher rate of release, for example. Where the therapeutic is administered by inhalation, the rate may be increased by administering additional boluses, a more concentrated bolus, or a faster-release bolus, for example. Other modes of administration (via subcutaneous injection pump, suppository, etc.) can be modulated in analogous fashions, and decreasing the rate of administration can be accomplished by doing the opposite of an action that would increase the rate of administration of the therapeutic.

In some embodiments, the subject has received an ACE inhibitor within a preceding period of time. The ACE inhibitor may be perindopril, captopril, enalapril, lisinopril, benazepril, fosinopril, moexipril, quinapril, trandolapril, and ramipril. In some embodiments, the ACE activity in the patient has been inhibited.

### Angiotensin Therapeutics

Angiotensin II are peptide hormones naturally produced by the body that regulates blood pressure via vasoconstriction and sodium reabsorption. Hemodynamic effects of angiotensin II administration have been the patient of numerous clinical studies, demonstrating significant effects on systemic and renal blood flow (Harrison-Bernard, L. M., The renal renin-angiotensin system. Adv Physiol Educ, 33(4):270 (2009)). Angiotensin II is a hormone produced by the renin angiotensin aldosterone system (RAAS) that modulates blood pressure via regulation of vascular smooth muscle tone and extracellular fluid homeostasis. Angiotensin II mediates its effects on the vasculature by inducing vasoconstriction and sodium retention, and so is the target of many therapies for hypertension. In addition to its systemic effects, angiotensin II has a pronounced effect on the efferent arterioles of the kidney, maintaining glomerular filtration when blood flow is decreased. Angiotensin II also regulates sodium reabsorption in the kidney by stimulating Na+/H+ exchangers in the proximal tubule and inducing the release of aldosterone and vasopressin (Harrison-Bernard, L. M., The renal renin-angiotensin system. Adv Physiol Educ, 33(4):270 (2009)).

The angiotensin II therapeutic that may be used for in the compositions and methods of this disclosure may be Asp-Arg-Val-Tyr-Ile-His-Pro-Phe (SEQ ID NO: 1) also called 5-isoleucine angiotensin II. SEQ ID NO: 1 is an octa-peptide naturally present in humans and other species, such as equines, hogs, etc. Isoleucine may be substituted by valine to result in 5-valine angiotensin II, Asp-Arg-Val-Tyr-Val-His-Pro-Phe (SEQ ID NO: 2). Other angiotensin II analogues such as [Asn¹-Phe⁴]-angiotensin II (SEQ ID NO: 3), hexapeptide Val-Tyr-Ile-His-Pro-Phe (SEQ ID NO: 4), nonapeptide Asn-Arg-Val-Tyr-Tyr-Val-His-Pro-Phe (SEQ ID NO: 5), [Asn¹-Ile⁵-Ile⁸]-angiotensin II (SEQ ID NO: 6), [Asn¹-Ile⁵-Ala⁸]-angiotensin II (SEQ ID NO: 7), and [Asn¹-diiodoTyr⁴-Ile⁵]-angiotensin II (SEQ ID NO: 8) may also be used. Angiotensin II may be synthesized, for example, by solid phase peptide synthesis to incorporate modifications, such as C-terminal amidation. The term “angiotensin II”, without further specificity, is intended to refer to any of these various forms, as well as combinations thereof.

In some aspects, a composition comprising angiotensin II may be selected from 5-valine angiotensin II, 5-valine angiotensin II amide, 5-L-isoleucine angiotensin II, and 5-L-isoleucine angiotensin II amide, or a pharmaceutically acceptable salt thereof, preferably manufactured under current good manufacturing conditions (cGMP). In some aspects, the composition may include different forms of angiotensin II in different percentages, e.g., a mixture of hexapeptide and nonapeptide angiotensin. The composition comprising angiotensin II may be suitable for parenteral administration, e.g., for injection or intravenous infusion.

The sequence of angiotensin II used in the compositions and methods disclosed herein may be homologous to the sequences of angiotensin II described above. In certain aspects, the invention includes isolated, synthetic, or recombinant amino acid sequences that are at least 80%, 85%, 90%, 95%, 97%, 98%, 99% or 100% identical to SEQ ID NO: 1, 2, 3, 4, 5, 6, 7, and/or 8. Any such variant sequences may be used in place of an angiotensin II as described in the preceding paragraph.

### Doses of the Therapeutically Effective Substance

In general, angiotensin II can increase blood pressure, and patients who are hypotensive may require larger doses to exhibit pressor responses similar to those observed in normal patients. The composition including the angiotensin therapeutic can be administered at a rate sufficient to achieve an increase in blood pressure of at least about 10-15 mm Hg and optionally for at least angiotensin therapeutic administered may be varied in response to changes in other physiological parameters such as renal vascular resistance, renal blood flow, filtration fractions, mean arterial pressure, etc. For example, the rate of administration of the angiotensin therapeutic may start from about 2 ng/kg/min to about 20 ng/kg/min and is increased based on the measured renin levels, PRA, mean arterial pressure (“MAP”). In some embodiments, the rate of administration may be increased such that the patient's renin levels and/or PRA fall below a threshold value. In some aspects, the rate of administration may be increased such that the MAP does not exceed about 70 mm Hg, about 80 mm Hg, about 90 mm Hg, about 100 mm Hg, about 110 mm Hg, etc. For example, a patient may be coupled to a monitor that provides continuous, periodic, or occasional measurements of MAP during some or all of the course of treatment. The rate of administration may be modulated manually (e.g., by a physician or nurse) or automatically (e.g., by a medical device capable of modulating delivery of the composition in response to MAP values received from the monitor) to maintain the patient's MAP within a desired range (e.g., 80-110 mm Hg) or below a desired threshold, e.g., as set forth above.

The composition including the angiotensin therapeutic may be administered over a period of time selected from at least 8 hours; at least 24 hours; and from 8 hours to 24 hours. The composition including the angiotensin therapeutic may be administered continuously for at least 2-6 days, such as 2-11 days, continuously for 2-6 days, for 8 hours a day over a period of at least 2-6 days, such as 2-11 days. A weaning period (from several hours to several days) may be beneficial after prolonged infusion.

The composition including the angiotensin therapeutic may further include one or more additional pharmaceutical agent. For example, angiotensin II may be administered with albumin. The quantity of the additional pharmaceutical agent administered may vary depending on the cumulative therapeutic effect of the treatment including the angiotensin therapeutic and the additional pharmaceutical agent. For example, the quantity of albumin administered may be 1 gram of albumin per kilogram of body weight given intravenously on the first day, followed by 20 to 40 grams daily. Yet other additional pharmaceutical agents may be any one or more of midodrine, octreotide, somatostatin, vasopressin analogue ornipressin, terlipressin, pentoxifylline, acetylcysteine, norepinephrine, misoprostol, etc. In some aspects, other natriuretic peptides may also be used in combination with the angiotensin therapeutic to remedy the impairment of sodium excretion associated with diseases discussed above. For example, natriuretic peptides may include any type of atrial natriuretic peptide (ANP), brain natriuretic peptide (BNP), C-type natriuretic peptide (CNP), and/or dendroaspis natriuretic peptide, etc. Several diuretic compounds may be used in combination with the angiotensin therapeutic to induce urine output. For example any one or more of the xanthines such as caffeine, theophylline, theobromine; thiazides such as bendroflumethiazide, hydrochlorothiazide; potassium-sparing diuretics such as amiloride, spironolactone, triamterene, potassium canrenoate; osmotic diuretics such as glucose (especially in uncontrolled diabetes), mannitol; loop diuretics such as bumetanide, ethacrynic acid, furosemide, torsemide; carbonic anhydrase inhibitors such as acetazolamide, dorzolamide; Na—H exchanger antagonists such as dopamine; aquaretics such as goldenrod, juniper; arginine vasopressin receptor 2 antagonists such as amphotericin B, lithium citrate; acidifying salts such as CaCl₂, NH₄Cl; ethanol, water, etc. may be used in combination with the angiotensin therapeutic to treat the patient. The list of additional pharmaceutical agents described above is merely illustrative and may include any other pharmaceutical agents that may be useful for the treatment of hypotension and related conditions.

### Excipients

The pharmaceutical compositions of the present invention may also contain diluents, fillers, salts, buffers, stabilizers, solubilizers, and other materials well known in the art. The term “pharmaceutically acceptable carrier” refers to a non-toxic carrier that may be administered to a patient, together with a therapeutically effective substance (such as angiotensin II) of this invention, and which does not destroy the pharmacological activity of the therapeutically effective substance. The term “pharmaceutically acceptable” means a non-toxic material that does not interfere with the effectiveness of the biological activity of the active ingredient(s). The characteristics of the carrier will depend on the route of administration. The term “excipient” refers to an additive in a formulation or composition that is not a pharmaceutically active ingredient.

One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to empirically determine which excipients, if any, to include in the compositions of the invention. Excipients of the invention may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some aspects, it may be beneficial to include a pharmaceutically acceptable carrier in the compositions of the invention.

### Solubilizing Agents

In some aspects, it may be beneficial to include a solubilizing agent in the compositions of the invention. Solubilizing agents may be useful for increasing the solubility of any of the components of the formulation or composition, including a therapeutically effective substance or an excipient. The solubilizing agents described herein are not intended to constitute an exhaustive list, but are provided merely as exemplary solubilizing agents that may be used in the compositions of the invention. In certain aspects, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

pH-Adjusting Agents

In some aspects, it may be beneficial to adjust the pH of the compositions by including a pH-adjusting agent in the compositions of the invention. Modifying the pH of a formulation or composition may have beneficial effects on, for example, the stability or solubility of a therapeutically effective substance, or may be useful in making a formulation or composition suitable for parenteral administration. pH-adjusting agents are well known in the art. Accordingly, the pH-adjusting agents described herein are not intended to constitute an exhaustive list, but are provided merely as exemplary pH-adjusting agents that may be used in the compositions of the invention. pH-adjusting agents may include, for example, acids and bases. In some aspects, a pH-adjusting agent includes, but is not limited to, acetic acid, hydrochloric acid, phosphoric acid, sodium hydroxide, sodium carbonate, and combinations thereof.

The pH of the compositions of the invention may be any pH that provides desirable properties for the formulation or composition. Desirable properties may include, for example, therapeutically effective substance (e.g., angiotensin II) stability, increased therapeutically effective substance retention as compared to compositions at other pHs, and improved filtration efficiency. In some aspects, the pH of the compositions of the invention may be from about 3.0 to about 9.0, e.g., from about 5.0 to about 7.0. In particular aspects, the pH of the compositions of the invention may be 5.5±0.1, 5.6±0.1, 5.7±0.1, 5.8±0.1, 5.9±0.1, 6.0±0.1, 6.1±0.1, 6.2±0.1, 6.3±0.1, 6.4±0.1, or 6.5±0.1.

### Buffers

In some aspects, it may be beneficial to buffer the pH by including one or more buffers in the compositions. In certain aspects, a buffer may have a pKa of, for example, about 5.5, about 6.0, or about 6.5. One of skill in the art would appreciate that an appropriate buffer may be chosen for inclusion in compositions of the invention based on its pKa and other properties. Buffers are well known in the art. Accordingly, the buffers described herein are not intended to constitute an exhaustive list, but are provided merely as exemplary buffers that may be used in the compositions of the invention. In certain aspects, a buffer may include one or more of the following: Tris, Tris HCl, potassium phosphate, sodium phosphate, sodium citrate, sodium ascorbate, combinations of sodium and potassium phosphate, Tris/Tris HCl, sodium bicarbonate, arginine phosphate, arginine hydrochloride, histidine hydrochloride, cacodylate, succinate, 2-(N-morpholino)ethanesulfonic acid (MES), maleate, bis-tris, phosphate, carbonate, and any pharmaceutically acceptable salts and/or combinations thereof.

### Surfactants

In some aspects, it may be beneficial to include a surfactant in the compositions of the invention. Surfactants, in general, decrease the surface tension of a liquid composition. This may provide beneficial properties such as improved ease of filtration. Surfactants also may act as emulsifying agents and/or solubilizing agents. Surfactants are well known in the art. Accordingly, the surfactants described herein are not intended to constitute an exhaustive list, but are provided merely as exemplary surfactants that may be used in the compositions of the invention. Surfactants that may be included include, but are not limited to, sorbitan esters such as polysorbates (e.g., polysorbate 20 and polysorbate 80), lipopolysaccharides, polyethylene glycols (e.g., PEG 400 and PEG 3000), poloxamers (i.e., pluronics), ethylene oxides and polyethylene oxides (e.g., Triton X-100), saponins, phospholipids (e.g., lecithin), and combinations thereof.

### Tonicity-Adjusting Agents

In some aspects, it may be beneficial to include a tonicity-adjusting agent in the compositions of the invention. The tonicity of a liquid composition is an important consideration when administering the composition to a patient, for example, by parenteral administration. Tonicity-adjusting agents, thus, may be used to help make a formulation or composition suitable for administration. Tonicity-adjusting agents are well known in the art. Accordingly, the tonicity-adjusting agents described herein are not intended to constitute an exhaustive list, but are provided merely as exemplary tonicity-adjusting agents that may be used in the compositions of the invention. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

### Stabilizing Agents

In some aspects, it may be beneficial to include a stabilizing agent in the compositions of the invention. Stabilizing agents help increase the stability of a therapeutically effective substance in compositions of the invention. This may occur by, for example, reducing degradation or preventing aggregation of a therapeutically effective substance. Without wishing to be bound by theory, mechanisms for enhancing stability may include sequestration of the therapeutically effective substance from a solvent or inhibiting free radical oxidation of the anthracycline compound. Stabilizing agents are well known in the art. Accordingly, the stabilizing agents described herein are not intended to constitute an exhaustive list, but are provided merely as exemplary stabilizing agents that may be used in the compositions of the invention. Stabilizing agents may include, but are not limited to, emulsifiers and surfactants.

### Routes of Delivery

The compositions of the invention can be administered in a variety of conventional ways. In some aspects, the compositions of the invention are suitable for parenteral administration. These compositions may be administered, for example, intraperitoneally, intravenously, intrarenally, or intrathecally. In some aspects, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

Throughout this specification, the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably. The terms “patient” and “individual” are used interchangeably and refer to either a human or a non-human animal. These terms include mammals such as humans, primates, livestock animals (e.g., bovines, porcines), companion animals (e.g., canines, felines) and rodents (e.g., mice, rabbits and rats).

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

### Exemplification

Angiotensin II therapy increases mean arterial pressure in patients with catecholamine-resistant vasodilatory shock. The analysis below of serum samples of patients treated with angiotensin II (see Table 1 and Tables 2-4 for renin specific data) demonstrates that serum renin concentration is markedly elevated in some patients with catecholamine-resistant vasodilatory shock. It also demonstrates that elevated serum renin may identify a subset of patients who gain a survival advantage when treated with angiotensin II. This finding supports a new approach to the treatment of catecholamine-resistant vasodilatory shock.

Serum samples were analyzed from patients enrolled in the Angiotensin II for the Treatment of High-Output Shock (ATHOS-3) trial for renin, angiotensin I, and angiotensin II levels prior to the start of administration of angiotensin II or placebo and after 3 hours. Baseline serum renin concentration (normal range: 2.13-58.78 pg/ml) was above the upper limits of normal (ULN) in 194/255 (76%) study patients with a median renin concentration of 172.7 pg/mL (interquartile range [IQR]: 60.7-440.6 pg/mL), approximately three-fold higher than ULN. Renin levels correlated positively with angiotensin I/angiotensin II ratios (r=0.39; P<0.001). At 3 hours after initiation of angiotensin II therapy, there was a 54.3% reduction (IQR: 37.9%-66.5% reduction) in renin concentration compared with a 14.1% reduction (IQR: 37.6% reduction to 5.1% increase) with placebo (P<0.0001). In patients with renin concentrations above the study population median, angiotensin II significantly reduced 28-day mortality to 28/55 (50.9%) patients compared with 51/73 patients (69.9%) treated with placebo (unstratified hazard ratio: 0.56; 95% confidence interval: 0.35-0.88; P=0.012) (p=0.048 for the interaction). This analysis supports a conclusion that serum renin concentration is markedly elevated in CRVS and identifies patients who gain a survival advantage when treated with angiotensin II.

TABLE 1
Demographics and Clinical Characteristics of Study Patients

| | Study Population Below the | | | | | Study Population Above the | | | | |
|---|---|---|---|---|---|---|---|---|---|---|
| | Median Renin | | | | | Median Renin | | | | |
| | Treatment with | | Treatment with | | | Treatment with | | Treatment with | | |
| | Placebo | | Angiotensin II | | | Placebo | | Angiotensin II | | |
| | N = 63 | | N = 64 | | P | N = 73 | | N = 55 | | P |
| Age, years, | 66 | (53-75) | 61.5 | (51-74) | 0.51 | 63 | (51-75) | 62 | (50-72) | 0.66* |
| Gender | | | | | 0.20 | | | | | 0.21^{†} |
| Female | 20 | (31.7%) | 28 | (43.8%) | | 26 | (35.6%) | 26 | (47.3%) | |
| Male | 43 | (68.3%) | 36 | (56.3%) | | 47 | (64.4%) | 29 | (52.7%) | |
| Race | | | | | 1.00 | | | | | 0.20^{†} |
| White | 52 | (82.5%) | 53 | (82.8%) | | 54 | (74.0%) | 46 | (83.6%) | |
| Nonwhite | 11 | (17.5%) | 11 | (17.2%) | | 19 | (26.0%) | 9 | (16.4%) | |
| Baseline | 2.3 | (1.8-2.8) | 2.2 | (1.6-2.6) | 0.21 | 2.3 | (1.9-2.7) | 2.3 | (1.9-2.7) | 0.75* |
| albumin (g/dL) | | | | | | | | | | |
| Median (IQR) | | | | | | | | | | |
| Baseline MELD | 22 | (15-25) | 20 | (14-25) | 0.26 | 23 | (20-28) | 22 | (18-26) | 0.09* |
| Median (IQR) | | | | | | | | | | |
| Baseline MAP | 66.7 | (64.0-68.7) | 66.0 | (63.7-67.9) | 0.40 | 66.3 | (62.3-68.0) | 66.7 | (63.3-69.7) | 0.28* |
| (mm Hg) | | | | | | | | | | |
| Median (IQR) | | | | | | | | | | |
| Baseline APACE | 29 | (20-34) | 27 | (22-33) | 0.70 | 31 | (25-36) | 28 | (22-34) | 0.14* |
| II score | | | | | | | | | | |
| Median (IQR) | | | | | | | | | | |
| ARDS^{#} | 17 | (27.0%) | 17 | (27.0%) | 1.0 | 28 | (38.4%) | 13 | (23.6%) | 0.088 |
| Medical history, | 56 | (88.9%) | 50 | (78.1%) | 0.15 | 57 | (78.1%) | 42 | (76.4%) | 0.83^{†} |
| sepsis | | | | | | | | | | |
| Recent ARB | 2 | (3.2%) | 4 | (6.3%) | 0.68 | 7 | (9.6%) | 3 | (5.5%) | 0.51 |
| Exposure | | | | | | | | | | |
| Recent ACEi | 5 | (7.9%) | 2 | (3.1%) | 0.27 | 8 | (11.0%) | 10 | (18.2%) | 0.31 |
| Severe AKI | 14 | (22.2%) | 18 | (28.1%) | 0.5 | 41 | (56.2%) | 21 | (38.2%) | 0.05 |
| ESRD | 2 | (3.2%) | 4 | (6.3%) | 0.6 | 1 | (1.4%) | 2 | (3.6%) | 0.58 |
| Baseline NED | 0.29 | (0.22-0.49) | 0.32 | (0.22-0.5 | 0.40 | 0.40 | (0.29-0.69) | 0.36 | (0.23-0.50) | 0.06* |
| (ug/kg/min) | | | | | | | | | | |
| Median (IQR) | | | | | | | | | | |
| Angiotensin I | 85.6 | (36.70-173.00) | 133.0 | (44.10-356.00) | 0.15 | 602 | (238-1110) | 655 | (304.5-1375) | 0.45* |
| (pg/ml) | | | | | | | | | | |
| Angiotensin II | 52.4 | (24.60-137.00) | 98.2 | (24.50 -168.00) | 0.35 | 108 | (16.7-523) | 151 | (41.4-439) | 0.46* |
| (pg/ml) | | | | | | | | | | |
| Baseline | 1.25 | (0.79-2.39) | 1.34 | (0.91-2.59) | 0.70 | 3.41 | (1.17-10.39) | 3.01 | (1.17-12.43) | 0.96* |
| angiotensin | | | | | | | | | | |
| I/II ratio | | | | | | | | | | |
| Median (IQR) | | | | | | | | | | |

Definition of abbreviations: APACHE = Acute Physiology and Chronic Health Evaluation; ARDS = acute respiratory distress syndrome; ESRD = end-stage renal disease; IQR, interquartile range; MAP = mean arterial pressure; MELD = Model for End-stage Liver Disease; NED = norepinephrine equivalent dose.
ACE inhibitor exposure was determined by the presence of an ACE inhibitor in the medical chart within 7 days prior to study enrollment.
^{#}ARDS was determined by chest x-ray reading. Patients with ARDS noted on their chest x-ray during screening were denoted as having ARDS.
*Wilcoxon rank-sum test.
^{†}Fisher's exact test.

TABLE 2
Population Characteristics Based on Renin Sample Availability

| | Missing at | Available at | |
|---|---|---|---|
| Parameter | Baseline | Baseline | Overall |
| Age, years | 64(56-75) | 63(51-74) | 63(52-74) |
| Gender (female, %) | 41.6% | 39.2% | 39.8% |
| Race (%) | | | |
| White | 77.5% | 80.4% | 79.7% |
| Black | 10.1% | 10.6% | 10.5% |
| Asian | 5.6% | 3.9% | 4.4% |
| Native Hawaiian or | 0.0% | 0.4% | 0.3% |
| Pacific Islander | | | |
| American Indian or | 0.0% | 0.4% | 0.3% |
| Alaska Native | | | |
| Other | 6.7% | 4.3% | 4.9% |
| Albumin g/dL | 2.3(1.8-2.7) | 2.3(1.9-2.7) | 2.3(1.8-2.7) |
| Baseline ScvO₂, % | 75.9(73-82) | 77.3(73-83) | 77(73-83) |
| CVP mm Hg | 12(10-15) | 13(10-16) | 12(10-16) |
| Cardiac Index (L/min/m²) | 3.2(2.3-5.4) | 3.1(2.1-66) | 3.1(2.1-66) |
| MELD | 21(15-26) | 22(16-26) | 22(16-26) |
| Baseline MAP, mm Hg | 66.7 (64-69) | 66.3(63-68) | 66.3(64-69) |
| APACHE II Score | 26(22-32) | 28(23-34) | 27.5(22-33) |
| Baseline NED, mcg/kg/min | 0.33(0.23-0.56) | 0.34(0.23-0.57) | 0.34(0.23-0.56) |

Legend: All data is presented as either percent or median (IQR) as appropriate

TABLE 3
Missing Serum Renin Imputation

| | | |
|---|---|---|
| Parameter | Estimate | S.E. |

Results Available Data

| | | |
|---|---|---|
| Treatment with Angiotensin II | −0.298 | 0.17 |
| Renin Level | 0.0004 | 0.0001 |

With Imputation*

| | | |
|---|---|---|
| Treatment with Angiotensin II | −0.229 | 0.16 |
| Renin Level | 0.0004 | 0.0001 |

*A multiple imputation model was utilized to investigate the sensitivity of missing data on the renin association with Day 28 survival. The imputation model included baseline MAP, APACHII score, NED and Angiotensin I. The renin results were robust and do not suggest an impact from missing data.

### Table 4. Sensitivity Analyses: Multi-Variate Analyses

4A: Whole cohort: Renin Level, Treatment
Assignment, APACHE, NED, and MAP

| Characteristic | HR (95% CI), p-value | |
|---|---|---|
| Full model | | |
| Treatment Arm - LJPC-501 | 0.77 (0.54-1.08) | 0.1275 |
| Baseline MAP - <65 mmHg | 1.54 (1.08-2.19) | 0.0160 |
| Baseline APACHE II Score - >30 | 1.67 (1.19-2.35) | 0.0030 |
| Baseline NE Equivalent Dose - >=0.5 | 1.72 (1.20-2.46) | 0.0030 |
| ug/kg/min | | |
| Renin >= Population Median | 1.63 (1.16-2.30) | 0.0049 |
| Step-wise model | | |
| Treatment Arm - LJPC-501 | 0.77 (0.54-1.08) | 0.1275 |
| Baseline MAP - <65 mmHg | 1.54 (1.08-2.19) | 0.0160 |
| Baseline APACHE II Score - >30 | 1.67 (1.19-2.35) | 0.0030 |
| Baseline NE Equivalent Dose - >=0.5 | 1.72 (1.20-2.46) | 0.0030 |
| ug/kg/min | | |
| Renin >= Population Median | 1.63 (1.16-2.30) | 0.0049 |

4B: Study Population below Median Renin with
Treatment Assignment, APACHE, NED, and MAP

| Characteristic | HR (95% CI), p-value | |
|---|---|---|
| Full model | | |
| Treatment Arm - LJPC-501 | 1.08 (0.64-1.82) | 0.7602 |
| Baseline MAP - <65 mmHg | 1.32 (0.76-2.32) | 0.3254 |
| Baseline APACHE II Score - >30 | 1.19 (0.69-2.04) | 0.5266 |
| Baseline NE Equivalent Dose - >=0.5 | 1.61 (0.91-2.86) | 0.1047 |
| ug/kg/min | | |
| Step-wise model | | |
| Treatment Arm - LJPC-501 | 1.11 (0.66-1.86) | 0.7001 |

4C: Study Population above Median Renin with
Treatment Assignment, APACHE, NED, and MAP

| Characteristic | HR (95% CI), p-value | |
|---|---|---|
| Full model | | |
| Treatment Arm - LJPC-501 | 0.62 (0.39-0.98) | 0.0423 |
| Baseline MAP - <65 mmHg | 1.66 (1.06-2.62) | 0.0279 |
| Baseline APACHE II Score - >30 | 2.03 (1.29-3.19) | 0.0023 |
| Baseline NE Equivalent Dose - >=0.5 | 1.78 (1.13-2.83) | 0.0137 |
| ug/kg/min | | |
| Step-wise model | | |
| Treatment Arm - LJPC-501 | 0.62 (0.39-0.98) | 0.0423 |
| Baseline MAP - <65 mmHg | 1.66 (1.06-2.62) | 0.0279 |
| Baseline APACHE II Score - >30 | 2.03 (1.29-3.19) | 0.0023 |
| Baseline NE Equivalent Dose - >=0.5 | 1.78 (1.13-2.83) | 0.0137 |
| ug/kg/min | | |

4D: Whole cohort by Renin Level, Treatment Assignment,
APACHE, NED, and MAP stratified by Treatment Assignment

| Characteristic | HR (95% CI), p-value | |
|---|---|---|
| Full model | | |
| Treatment Arm - LJPC-501 | 1.08 (0.64-1.81) | 0.7734 |
| Baseline MAP - <65 mmHg | 1.54 (1.09-2.19) | 0.0156 |
| Baseline APACHE II Score - >30 | 1.66 (1.18-2.33) | 0.0037 |
| Baseline NE Equivalent Dose - >=0.5 | 1.69 (1.18-2.42) | 0.0039 |
| ug/kg/min | | |
| Renin >= Population Median | 2.12 (1.34-3.38) | 0.0014 |
| Treatment * Renin >= Population | 0.54 (0.27-1.09) | 0.0875 |
| Median | | |
| Step-wise model | | |
| Treatment Arm - LJPC-501 | 0.77 (0.54-1.08) | 0.1275 |
| Baseline MAP - <65 mmHg | 1.54 (1.08-2.19) | 0.0160 |
| Baseline APACHE II Score - >30 | 1.67 (1.19-2.35) | 0.0030 |
| Baseline NE Equivalent Dose - >=0.5 | 1.72 (1.20-2.46) | 0.0030 |
| ug/kg/min | | |
| Renin >= Population Median | 1.63 (1.16-2.30) | 0.0049 |

### Introduction

Vasodilatory shock requiring vasopressor therapy is associated with poor outcomes. In particular, catecholamine-resistant vasodilatory shock (CRVS), in which hypotension persists despite the use of high-dose vasopressors, carries a 50% to 80% mortality. In these patients, first-line vasopressor therapy, which includes norepinephrine followed by epinephrine or vasopressin, is often inadequate to achieve target mean arterial pressure (MAP).

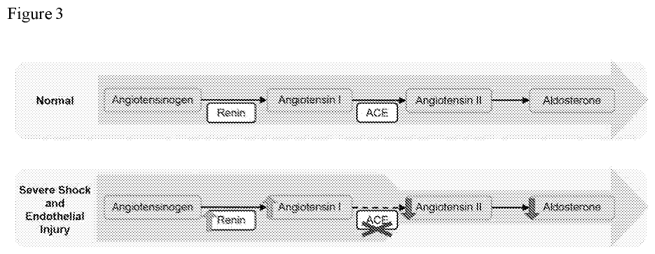

Angiotensin II is an integral part of the renin-angiotensin-aldosterone system (RAAS) (FIG. 3). Angiotensin I, the precursor of angiotensin II, is cleaved from angiotensinogen by renin, a proteolytic enzyme released by juxtaglomerular cells in response to sympathetic nerve activation, hypotension, or decreased sodium delivery to the distal tubule. Renin release is inhibited by high angiotensin II generation and promoted by low angiotensin II generation via a biofeedback loop involving the angiotensin type 1 receptor. As such, renin levels are increased when there is insufficient activation of the angiotensin II type 1 receptor. This can be caused by decreased angiotensin II generation or angiotensin II receptor blockade. In this regard, the primary pathway for angiotensin II generation is via angiotensin-converting enzyme (ACE), an endothelial membrane-bound enzyme that cleaves angiotensin I to angiotensin II. Endothelial injury accompanying CRVS should decrease ACE function, increase angiotensin I to angiotensin II ratios, and promote renin release. High renin levels would potentially identify patients most likely to benefit from angiotensin II therapy. In support of this theory, high ratios of angiotensin I to angiotensin II correlate with negative outcomes in such patients. Moreover, in a study of patients with sepsis, reduced angiotensin II and ACE levels outperformed established illness-severity scores in predicting outcomes.

Renin levels, which are expected to increase in CRVS patients with ACE dysfunction and high angiotensin I to angiotensin II levels, could be used to identify such high-risk patients. Laboratory assays that measure renin levels are inexpensive, FDA approved, have demonstrated established performance, and can be easily set up by most laboratories; importantly, measurement of renin levels outperforms that of maximum serum lactate levels as a predictor of mortality in the intensive care unit (ICU). Accordingly, we hypothesized that plasma renin levels would be high in patients with CRVS, would correlate with angiotensin I to angiotensin II ratios, and when elevated would help to identify those patients most likely to benefit from intravenous angiotensin II therapy.

### Methods

### Study Design and Patients

The Angiotensin II for the Treatment of High-Output Shock 3 (ATHOS-3) study has been previously described—NCT02338843. In brief, adults with vasodilatory shock despite volume resuscitation with ≥25 mL/kg and high-dose vasopressors (defined as a norepinephrine equivalent [NED] of greater than 0.2 mcg/kg/min) were randomly assigned 1:1 to receive synthetic human angiotensin II (La Jolla Pharmaceutical Company, San Diego, Calif.) or saline placebo plus standard vasopressors. Randomization was stratified according to MAP at screening and Acute Physiology and Chronic Health Evaluation II (APACHE II) score.

Study drug infusion started at 20 ng/kg/min and was adjusted during the first 3 hours to increase the MAP to ≥75 mm Hg while keeping other vasopressors constant. Thereafter, the study drug and other vasopressors were adjusted at the discretion of the treating ICU team to maintain a MAP between 65 and 75 mm Hg. At 48 hours, the infusion was discontinued according to a protocol-specified tapering process. However, continuation was allowed for up to 7 days at the discretion of the ICU team.

### Serum Renin, Angiotensin I, and Angiotensin II Measurement

Serum concentrations of renin, angiotensin I, and angiotensin II were measured after randomization but prior to administration of the study drug and at 3 hours after initiation of study drug.

### Statistical Analysis

Descriptive statistics with 95% confidence intervals (CIs) were used to summarize data according to treatment group. Differences between treatment groups were identified using the Wilcoxon rank-sum test or analysis of variance for continuous or ordinal variables and the chi-square or Fisher's exact test for discrete variables. Time-to-event data was summarized, including survival, using Kaplan-Meier estimates and compared them by log-rank test. Hazard ratios (HRs) are estimated from a proportional hazards model. A two-sided alpha level of 0.05 was used to test for differences in treatment outcomes without adjustments for multiplicity. Correlation coefficients were calculated for renin versus angiotensin I and the angiotensin I/II ratio. Correlation coefficients were also calculated for renin versus angiotensin I for hour 0 (baseline) and hour 3. For multivariate analyses, covariates that were utilized for study stratification (Model A), covariates that were different between groups defined by P<0.10 (Model B), and statistically significant covariates from Models A and B were included in a multivariate logistic regression (Model C). The dichotomized baseline covariates that were utilized for study stratification were MAP <65 mm Hg and APACHE II score >30.

Safety was evaluated by assessment of treatment-emergent adverse events, serious adverse events, and adverse event-related drug discontinuations. Data were analyzed with the use of SAS software, version 9.4 (SAS Institute, Cary, N.C.) and R version 3.5.3. (R Foundation).

### Additional Methods

Serum renin was measured with the DRG Renin ELISA Kit (DRG Instruments GmbH, Marburg, Germany). The DRG ELISA Kit is a solid-phase enzyme-linked immunosorbent assay based on the sandwich principle. The microtiter wells are coated with a monoclonal (mouse) antibody directed toward a unique antigenic site of the human active renin molecule. An aliquot of patient sample containing endogenous renin is rapidly thawed in a water bath and incubated in the coated well together with assay buffer. After incubation, unbound components are washed off. Finally, enzyme conjugate, a monoclonal anti-renin antibody conjugated with horseradish peroxidase, is added. After incubation, unbound enzyme conjugate is washed off. The amount of bound peroxidase is proportional to the concentration of renin in the sample. Having added the substrate solution, the intensity of color developed is proportional to the concentration of active renin in the patient sample. Personnel who conducted the assays were blinded to the study database and treatment allocation.

### Results

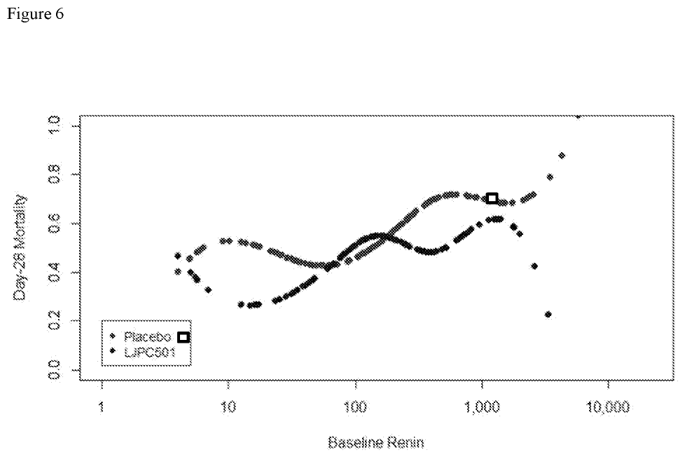

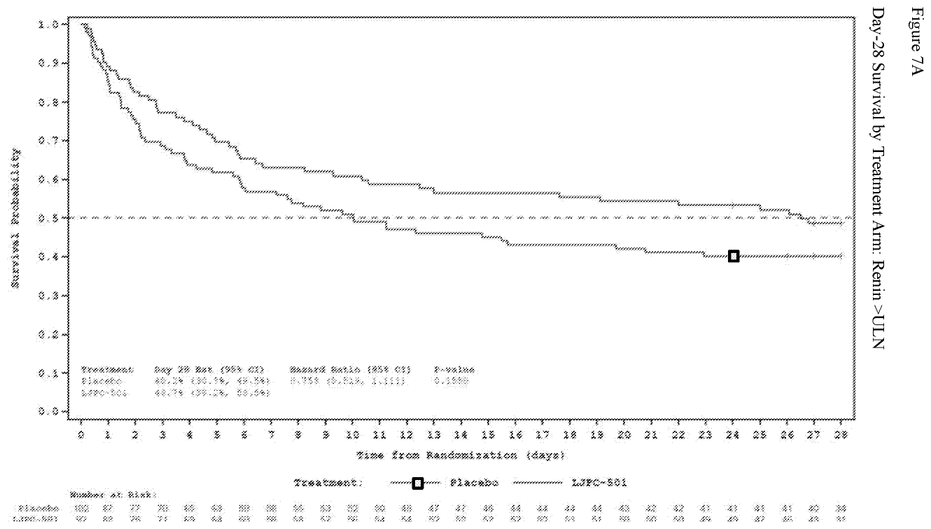

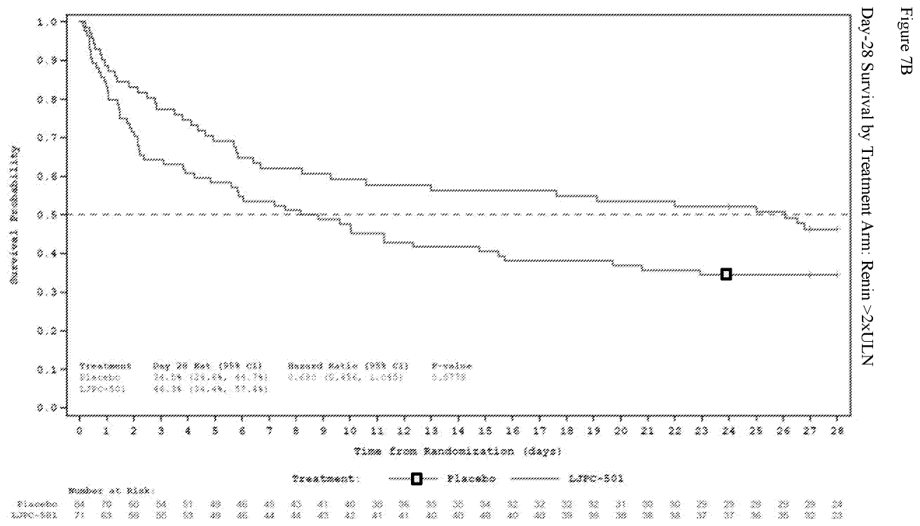

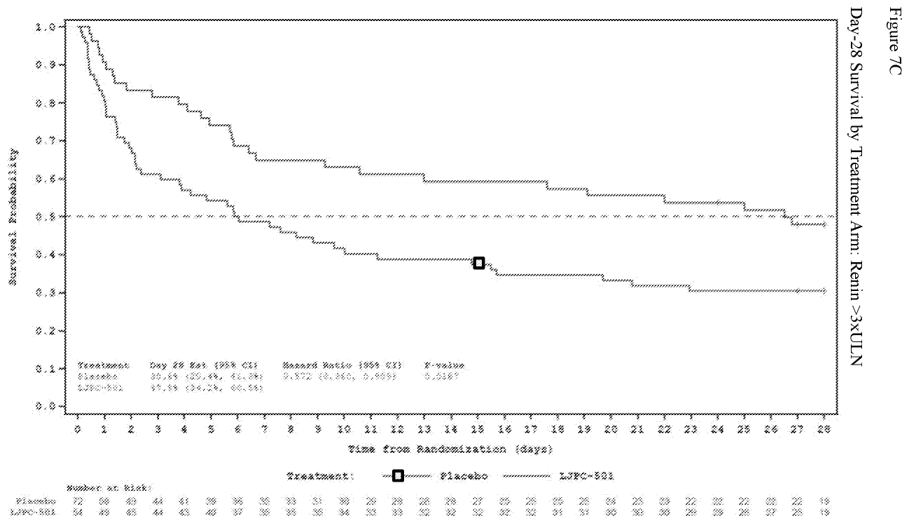

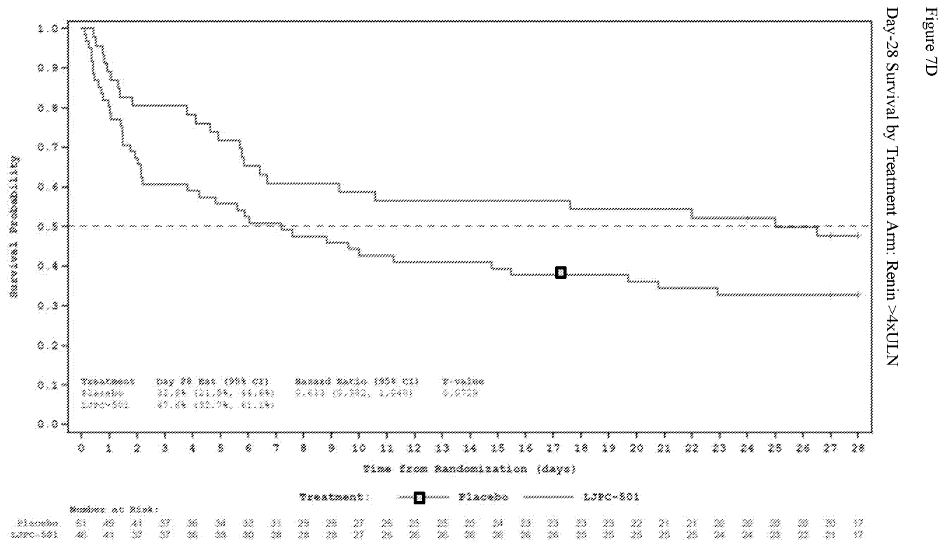

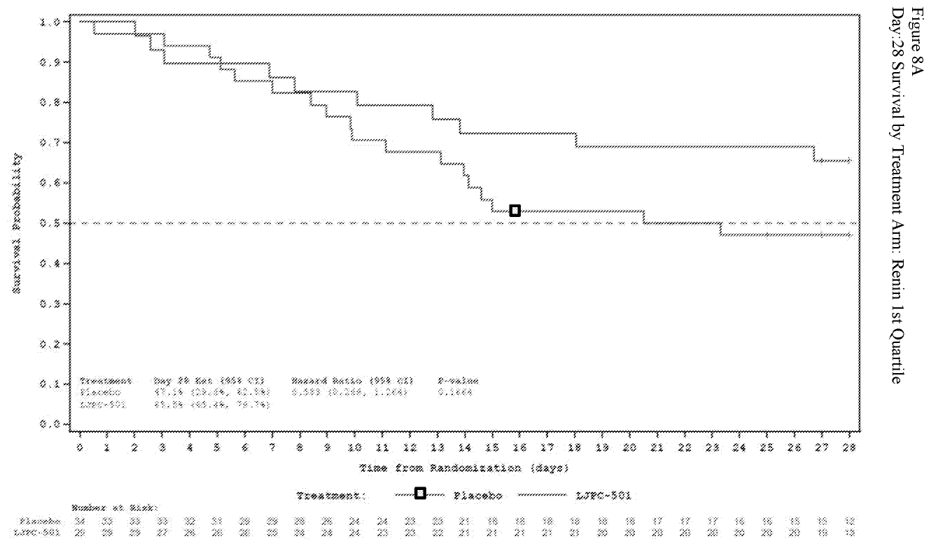

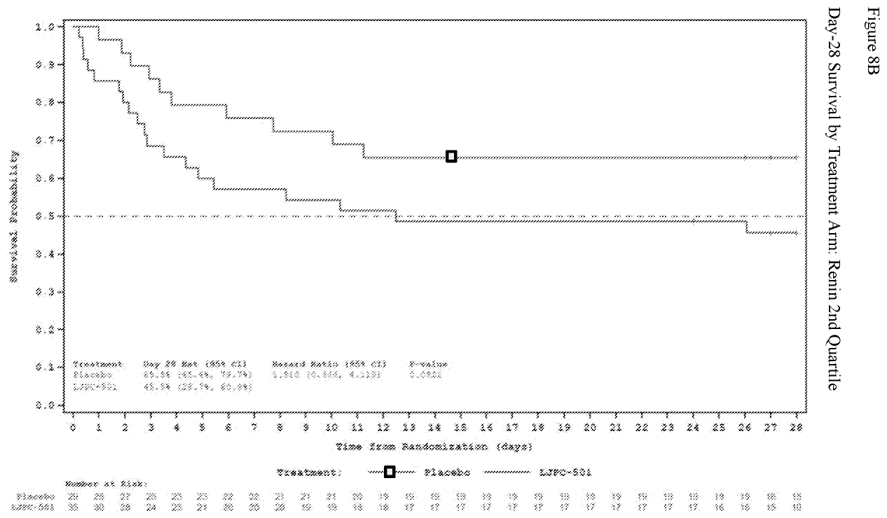

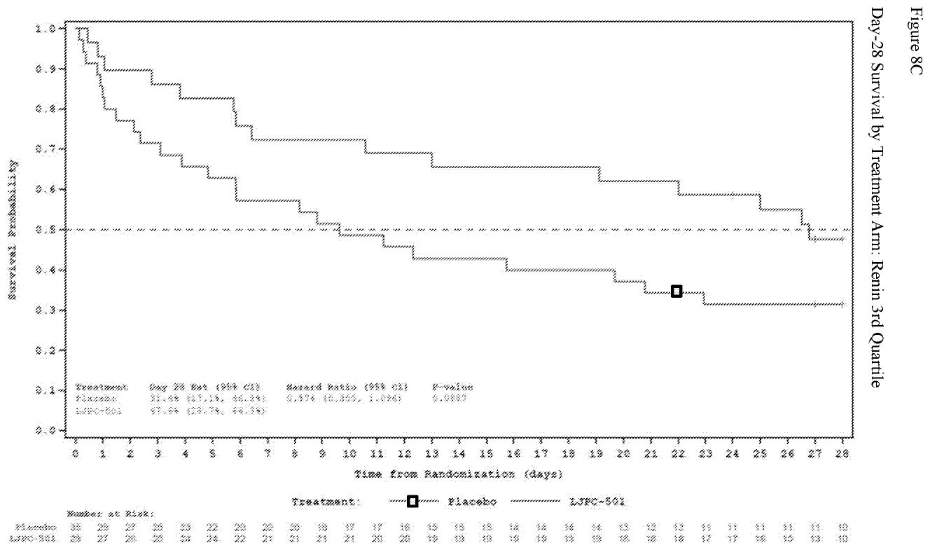

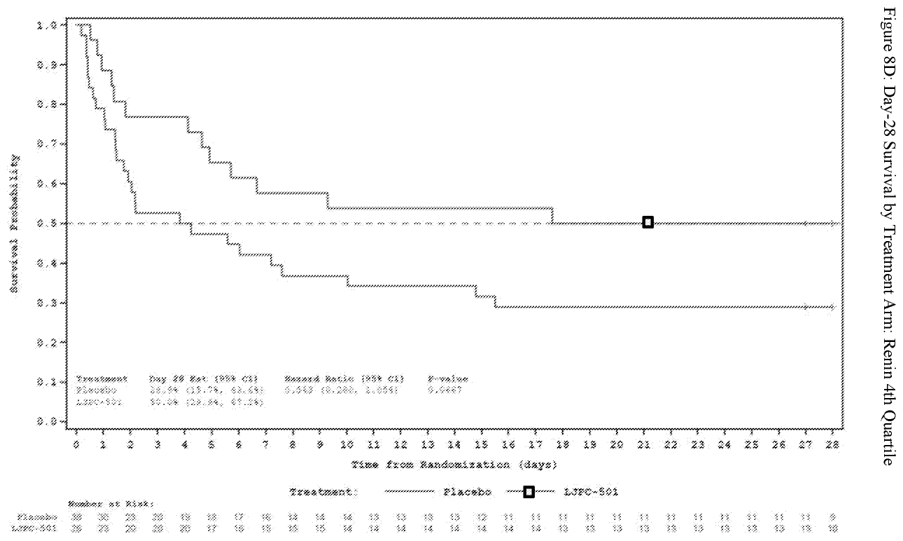

Of the 321 patients with CRVS studied in ATHOS-3, 255 (79.4%) had suitable serum samples available for analysis of renin concentrations at baseline (hour 0) (Tables 18, 2, and 3). In these patients, baseline serum renin concentrations were comparable between the angiotensin II and placebo arms and were elevated to levels above the upper limit of normal in 194 (76%) patients. The median serum renin concentration of the entire population was 172.7 pg/mL (interquartile range [IQR]: 60.7-440.6 pg/mL); this was approximately 3 times the upper limit of normal (ULN; 58.78 pg/ml). Table 5 and FIG. 6 further show baseline renin levels in patient subsets. In addition, renin and angiotensin I were also positively correlated at 3 hours of treatment (P<0.001).

TABLE 5
Serum Renin Levels in Various Study Sub-populations

| | Baseline Serum Renin |
|---|---|
| | median (IQR) |
| All Patients | 172.7 (60.7-440.6) |
| AKI Population^{1.} (n = 94) | 352.5 (115.9-785.4) |
| Medical History of ESRD (n = 9) | 138.3 (96.4-193.3) |
| Non AKI, non ESRD (n = 152) | 121.6 (40.6-343.6) |
| ARDS by Chest X-ray (n = 75) | 210.5 (107.8-563.2) |
| Non-ARDS by Chest X-ray (n = 179) | 148.6 (45.9-427.5) |
| Exposure to ACE Inhibitor (n = 25) | 346.9 (144.5-784.4) |
| No Exposure to ACE Inhibitor (n = 230) | 150.2 (55.4-427.5) |
| Exposure to ARB (n = 16) | 223.4 (121.8-382.0) |
| No Exposure to ARB (n = 239) | 162.2 (59.6-440.6) |

^{1.}No medical history of end-stage renal disease with dialysis at baseline

Table 6 shows patient demographic and clinical data dichotomized by median serum renin levels, and Table 1 shows these data further dichotomized by treatment group within each renin-level stratum. At baseline, patients with serum renin levels above the study population median were similar to those with levels below the study population median, except for baseline levels of angiotensin I, angiotensin II, angiotensin I/II ratio, and norepinephrine-equivalent dose, all of which were significantly higher in the former group.

TABLE 6
Mortality Sensitivity Analysis: High-renin and Low- Renin demarcated
by Multiple of the Upper Limit of Normal of the Renin Assay

| Cut-Off | HR | CI | P value |
|---|---|---|---|
| ULN | 0.76 | 0.519-1.111 | 0.1 |
| 2x ULN | 0.69 | 0.456-1.045 | 0.0 |
| 3x ULN | 0.57 | 0.360-0.909 | 0.0 |
| 4x ULN | 0.63 | 0.382-1.048 | 0.0 |

Legend: ULN = upper limit of normal

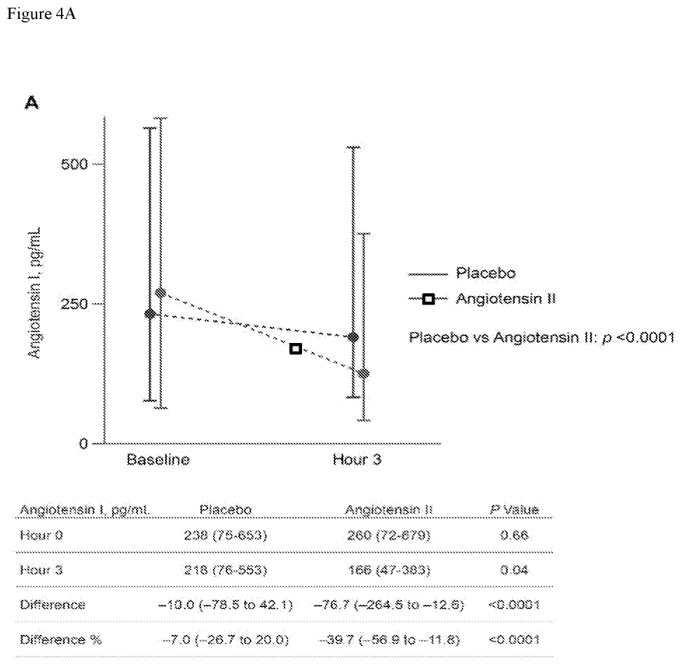

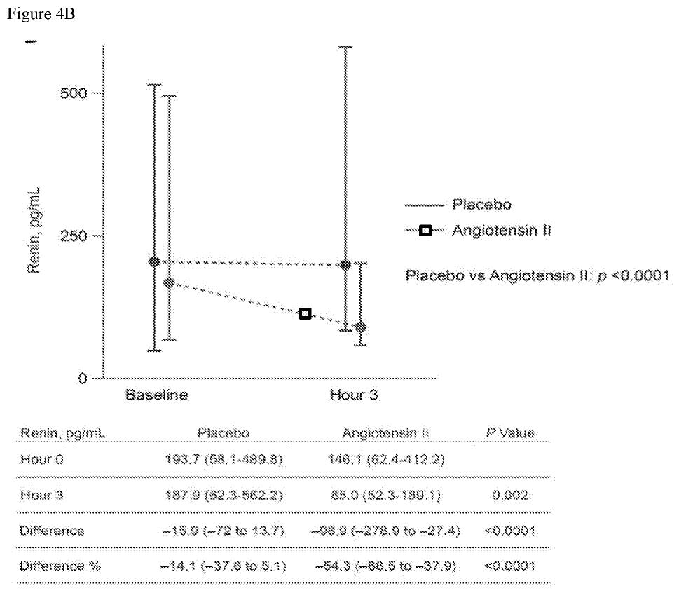

FIG. 4 shows serum levels of angiotensin I and renin in patients who had samples available at baseline and at 3 hours. Median baseline renin levels were similar among patients in the placebo group (193.7 [58.1-489.8] pg/ml) and patients in the angiotensin II group (146.1 [62.4-412.2] pg/ml) (P=0.4277). Patients treated with synthetic angiotensin II experienced a median reduction of 54.3% (IQR: 37.9%-66.5% reduction) in renin concentration compared with a median reduction of 14.1% (IQR: 37.6% reduction to 5.1% increase) in patients treated with placebo (P<0.0001) at hour 3. Median baseline levels of angiotensin I were also similar between treatment groups, and as was seen with serum renin levels, treatment with synthetic angiotensin II led to a significantly greater reduction in angiotensin I levels at hour 3 (40.8% median reduction [IQR: 13.4%-65.1% reduction]) compared to treatment with placebo (8.1% reduction [IQR: 29.9% reduction to 15.2% increase]) (P<0.0001).

### Survival by Baseline Serum Renin Levels

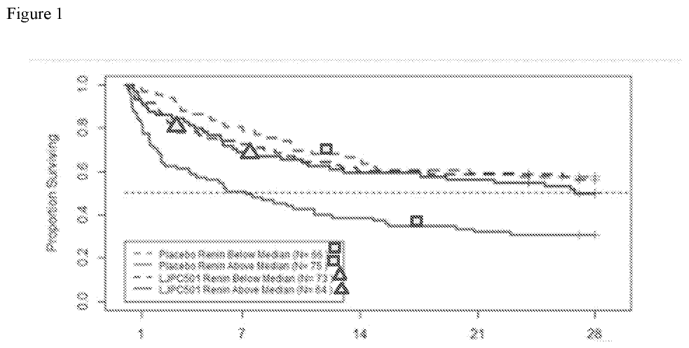

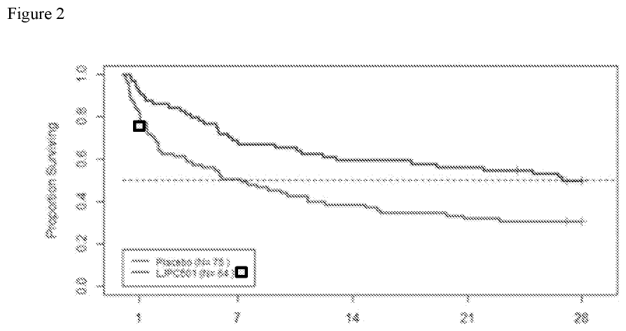

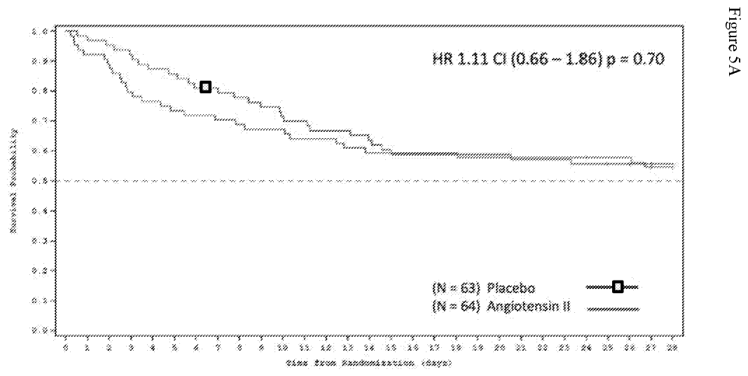

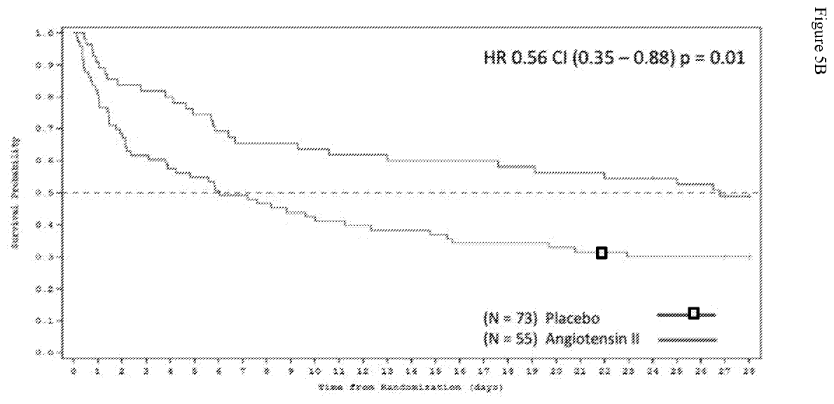

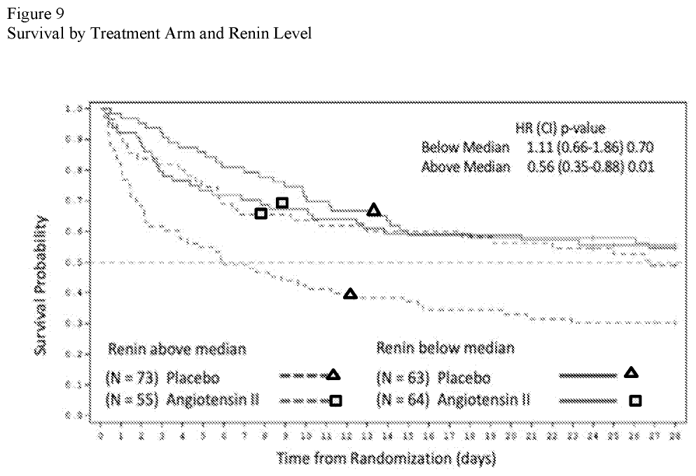

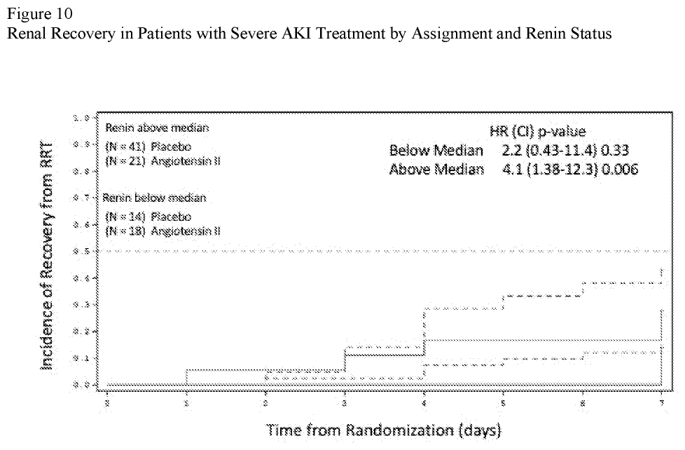

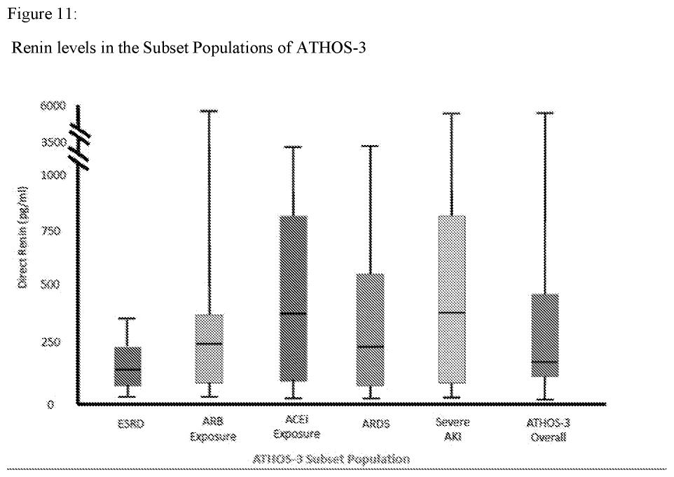

For patients with renin levels above the study population median, baseline characteristics were well balanced between the placebo and angiotensin II treatment arms. (Table 1). In spite of this balance, patients treated with placebo had a 28-day mortality rate of 69.9% compared with 50.9% in patients treated with angiotensin II (unstratified HR, 0.556; 95% CI, 0.35-0.88; P=0.0115) (Table 7, FIGS. 1 and 2, and Table 15 and Table 9; see also FIGS. 9, 10 and 14). In contrast, there was no statistically significant difference in 28-day mortality for those with serum renin levels below the study population median (Table 7 and FIG. 5).

TABLE 7A
Outcomes and Renin Level: Patients Below the Study Population Median Renin

| | Patients Below the Study Population Median Renin | | |
|---|---|---|---|
| | Treatment with | Treatment with | |
| | Placebo | Angiotensin II | |
| | N = 63 | N = 64 | P |
| 28-day mortality | 44% (33%-58%) | 45% (34%-58%) | 0.70 |
| Ventilator Liberation by Day 7 | 38% (27%-52%) | 27% (18%-40%) | 0.20 |
| (alive and vent free) | | | |
| RRT Liberation by Day 7 | 14% (4%-46%) | 28% (13%-54%) | 0.33 |
| (alive and RRT free) | | | |
| ICU Discharge by Day 28 | 52% (41%-65%) | 39% (28%-52%) | 0.13 |

TABLE 7B
Outcomes and Renin Level: Patients Above the Study Population Median Renin

| | Patients Above the Study Population Median Renin | | |
|---|---|---|---|
| | Treatment with | Treatment with | |
| | Placebo | Angiotensin II | |
| | N = 73 | N = 55 | P |
| 28-day mortality | 70% ( 59%-80%) | 51% (39%-65%) | 0.01 |
| Ventilator Liberation by Day 7 | 14% (8%-25%) | 28% (18%-43%) | 0.07 |
| (alive and vent free) | | | |
| RRT Liberation by Day 7 | 12% (5%-27%) | 43% (25%-66%) | 0.01 |
| (alive and RRT free) | | | |
| ICU Discharge by Day 28 | 22% (14%-33%) | 44% (32%-58%) | 0.02 |

TABLE 8
Day 28 Mortality by Baseline Renin

| | Placebo | Ang II | p-value |
|---|---|---|---|
| Renin | | | |
| <Pop Median | N = 66, 42.4% | N = 73, 43.9% | 0.06705 |
| >=Pop Median | N = 75, 69.3% | N = 64, 50.1% | 0.0064 |
| p-value [b] | 0.0002 | 0.5734 | |

TABLE 9
Day 28 Mortality Renin Greater than Population Median

| | Placebo | Ang II | p-value |
|---|---|---|---|
| Renin | | | |
| >=Pop Median | N = 75, 69.3% | N = 64, 50.1% | 0.0064 |

Multivariate logistic regression within the placebo treatment arm alone showed that, after adjusting for age, sex, APACHE II score, MAP, and norepinephrine-equivalent dose, elevated serum renin levels were independently associated with an increased risk of death (HR, 2.15; 95% CI, 1.35-3.42; P=0.0013 (Table 10). Moreover, in patients with a renin level above the study population median, multivariable analysis identified treatment with angiotensin II as associated with decreased risk of mortality (HR, 0.62; 95% CI, 0.39-0.98; P=0.0423) (Table 11). See also Tables 4 and 6 for sensitivity analyses of these multivariable analyses. FIG. 6 displays survival by serum renin level as a continuous variable.

TABLE 10
Multivariable Analysis for the Prediction
of Mortality: Placebo Arm Only

| | Hazard Ratio (95% CI) | P Value |
|---|---|---|
| Model A | | |
| Renin above population | 2.15 (1.35-3.42) | 0.0013 |
| median | | |
| Baseline APACHE II score >30 | 1.87 (1.19-2.93) | 0.0062 |
| Baseline MAP <65 mm Hg | 1.86 (1.18-2.93) | 0.0076 |
| Model B | | |
| Renin above the population | 2.50 (1.33-4.67) | 0.0042 |
| median | | |
| Baseline MELD ≥30 | 1.61 (0.90-2.86) | 0.1081 |
| Baseline APACHE II score >30 | 1.91 (1.19-3.07) | 0.0076 |
| Baseline Angiotensin I <253 | 1.74 (0.90-3.36) | 0.0980 |
| pg/mL | | |
| Baseline Angiotensin II <83.75 | 0.84 (0.45-1.55) | 0.5680 |
| pg/mL | | |
| Baseline Angiotensin I/II | 0.81 (0.46-1.44) | 0.4803 |
| ratio <1.63 | | |
| Baseline NED ≥0.5 ug/kg/min | 1.98 (1.20-3.27) | 0.0071 |
| Model C | | |
| Renin above population | 2.11 (1.33-3.36) | 0.0016 |
| median | | |
| Baseline APACHE II score >30 | 1.88 (1.20-2.95) | 0.0056 |
| Baseline MAP <65 mm Hg | 1.66 (1.04-2.66) | 0.0340 |
| Baseline NED ≥0.5 ug/kg/min | 1.60 (1.00-2.57) | 0.0518 |

Definition of abbreviations: APACHE = Acute Physiology and Chronic Health Evaluation; CI = confidence interval; MAP = mean arterial pressure; MELD = Model for End-Stage Liver Disease; NED = norepinephrine equivalent dose.
Model A includes treatment assignment and stratification variables. Model B includes covariates that were imbalanced by P < 0.10 in the population clinical characteristics. Model C includes the statistically significant variables from Model A and Model B.

TABLE 11
Multivariable Analysis for the Prediction of Mortality:
Patients With Elevated Baseline Renin Concentrations Only

| | Hazard Ratio (95% CI) | P Value |
|---|---|---|
| Model A | | |
| Treatment arm, angiotensin II | 0.58 (0.36-0.93) | 0.02 |
| Baseline APACHE II score >30 | 2.02 (1.29-3.18) | 0.002* |
| Baseline MAP <65 mm Hg | 1.76 (1.12-2.77) | 0.01 |
| Model B | | |
| Treatment arm, angiotensin II | 0.62 (0.38-0.99) | 0.04 |
| Baseline NED ≥0.5 ug/kg/min | 1.88 (1.19-2.98) | 0.007 |
| Baseline MELD ≥30 | 1.43 (0.81-2.50) | 0.21 |
| Model C | | |
| Treatment arm, angiotensin II | 0.62 (0.39-0.98) | 0.04 |
| Baseline APACHE II score >30 | 2.03 (1.29-3.19) | 0.002* |
| Baseline MAP <65 mm Hg | 1.66 (1.06-2.62) | 0.03 |
| Baseline NED ≥0.5 ug/kg/min | 1.78 (1.13-2.83) | 0.01 |

Definition of abbreviations: APACHE = Acute Physiology and Chronic Health Evaluation; CI = confidence interval; MAP = mean arterial pressure; MELD = Model for End-Stage Liver Disease; NED = norepinephrine equivalent dose.
*p < 0.01
Model A includes treatment assignment and stratification variables. Model B includes covariates that were imbalanced by P < 0.10 in the population clinical characteristics. Model C includes the statistically significant variables from Model A and Model B.

TABLE 12
Overall Study Cohort Demographics and Clinical Characteristics
by Study Population Renin Levels

| | Overall Study Cohort by Study Population | | |
|---|---|---|---|
| | Renin | Renin | |
| | Below | Above | |
| | Median | Median | |
| | N = 127 | N = 128 | P |
| Age, years, | 63 (51-74) | 62 (51-74) | 0.52* |
| median (IQR) | | | |
| Gender | | | |
| Female | 48 (37.8%) | 52 (40.6%) | 0.7008† |
| Male | 79 (62.2%) | 76 (59.4%) | |
| Race | | | |
| White | 105 (82.7%) | 100 (78.1%) | 0.4308† |
| Nonwhite | 22 (17.3%) | 28 (21.9%) | |
| Baseline albumin | 2.2 (1.8-2.8) | 2.3 (1.9-2.7) | 0.6115* |
| Baseline MELD | 21 (14-25) | 23 (18-27) | 0.0681* |
| Median (IQR) | | | |
| Baseline MAP (mm Hg) | 66.3 (63.7-68.3) | 66.3 (62.9-68.7) | 0.6779* |
| Median (IQR) | | | |
| Baseline APACHE II score | 27 (21-33) | 29 (24-35) | 0.0691* |
| Median (IQR) | | | |
| Medical history, ARDS | | | |
| No | 107 (84.3%) | 101 (78.9%) | 0.3328† |
| Yes | 20 (15.7%) | 27 (21.1%) | |
| Chest X-ray, ARDS | | | |
| No | 92 (73.0%) | 87 (68.0%) | 0.4108† |
| Yes | 34 (27.0%) | 41 (32.0%) | |
| Medical history, sepsis | | | |
| No | 21 (16.5%) | 29 (22.7%) | 0.2697† |
| Yes | 106 (83.5%) | 99 (77.3%) | |
| Severe AKI | | | |
| No | 95 (74.8%) | 66 (51.6%) | 0.0002 |
| Yes | 32 (25.2%) | 62 (48.4%) | |
| ESRD | | | |
| No | 121 (95.3%) | 125 (97.7%) | 0.3339 |
| Yes | 6 (4.7%) | 3 (2.3%) | |
| Baseline NED (ug/kg/min) | 0.30 (0.22-0.50) | 0.38 (0.25-0.60) | 0.0132* |
| Median (IQR) | | | |
| Angiotensin I (pg/ml) | 95.5 (39.2-275) | 631 (267-1310) | <0.0001* |
| Angiotensin II (pg/ml) | 64.6 (24.5-160) | 139.5 (28.2-513.5) | 0.0008 |
| Baseline angiotensin I/II ratio | 1.29 (0.83-2.59) | 3.10 (1.17-11.90) | <0.0001* |
| Median (IQR) | | | |

TABLE 13
Survival in Acute Kidney Injury and ARDS
Patient Subsets in High/Low Renin

| | High-Renin | Low renin |
|---|---|---|
| Population | N, HR (95% CI), | N, HR (95% CI), |
| (n) | p value | p value |
| LJPC 501 v. | N = 128, | N = 127, |
| Placebo (All) | 0.56 (0.35-0.88), 0.0115 | 1.11 (0.66-1.86), 0.7002 |
| LJPC 501 v. | N = 62, | N = 32, |
| Placebo (AKI) | 0.51 (0.26-0.99), 0.0411 | 0.55 (0.19-1.58), 0.2574 |
| LJPC 501 v. | N = 41, | N = 34, |
| Placebo (ARDS) | 0.49 (0.21-1.16), 0.0964 | 1.38 (0.59-3.26), 0.4583 |

### Other Outcomes of Interest

Table 7 shows rates of ventilator and RRT liberation, as well as ICU discharge according to treatment group in patients dichotomized by serum renin levels. Whereas there were no differences in these outcomes between treatment groups in patients with renin levels below the study population median, in patients with renin levels above the study population median, RRT liberation by day 7 was significantly greater in patients treated with angiotensin II (43%) compared to those treated with placebo (12%; P=0.01) (Table 7; Table 16, FIG. 5, Table 14, and Table 15), as was ICU discharge by day 28 (angiotensin II: 44%; placebo: 22%; P=0.02). The rate of ventilator liberation by day 7, however, did not differ significantly between treatment groups (angiotensin II: 28%; placebo: 14%; P=0.07).

TABLE 14
Renin

| | Placebo | Ang II | p-value |
|---|---|---|---|
| Baseline Renin | | | |
| N | 141 | 137 | 0.4360 |
| Mean (SD) | 468.2 (794.19) | 381.3 (595.08) | |
| Median (range) | 192.3 (4.0-5762.4) | 140.8 (4.0-3617.3) | |
| Hour 3 Renin | | | |
| N | 114 | 103 | 0.0046 |
| Mean (SD) | 491.0 (868.63) | 231.8 (416.91) | |
| Median (range) | 182.3 (9.5-6689.8) | 85.0 (4.0-2739.8) | |
| Change at Hour 3 | | | |
| N | 113 | 102 | <0.0001 |
| Mean (SD) | −46.4 (288.35) | −210.3 (307.43) | |
| Median (range) | −15.9 (−1584-1060.6) | −99.6 (−1574-496.2) | |
| % Change at Hour 3 | | | |
| N | 113 | 102 | <0.0001 |
| Mean (SD) | −11.2 (40.02) | −45.5 (38.59) | |
| Median (range) | −14.5 (−67.7-204.8) | −54.8 (−91.1-161.0) | |

[WL1]

TABLE 15
MAP Response at Hour 3 by Baseline Renin

| | Placebo | Ang II | p-value |
|---|---|---|---|
| Renin | | | |
| <Pop Median | 18/66 (27.3%) | 57/73 (78.1%) | <0.0001 |
| >=Pop Median | 16/75 (21.3%) | 39/64 (60.9%) | <0.0001 |
| p-value [b] | 0.4107 | 0.0288 | |

TABLE 16
Likelihood of Renal Recovery in Patients
with Severe AKI by Serum Renin Levels

| | LJPC 501 (Ang II) v. |
|---|---|
| | Placebo N, RR (95% CI), |
| All patients | N = 105*, |
| (severe AKI) | 2.902 (1.293-6.515) |
| Renin above | N = 62, |
| study median | 4.112 (1.376-12.29), 0.0061 |
| Renin below | N = 32, |
| study median | 2.215 (0.429-11.42), 0.3295 |

Severe AKI = Patients with AKI requiring RRT at the time of study enrollment. Renal recovery was assessed over the first 7 days.
*All patients even those without renin level available.

Table 17 shows the changes in cardiovascular (CV) and total SOFA scores between baseline and hour 48 according to renin status and treatment group. In the total ATHOS-3 population, the change in CV score was significantly greater in the angiotensin II compared to the placebo group, whereas the change in total SOFA score was not. As shown in Table 17 neither the change in CV SOFA nor the total SOFA score differed significantly between treatment groups in patients with serum renin levels below the study population median. In patients with serum renin levels above the study population median, the mean (±SD) change in CV SOFA score at 48 hours was significantly greater in the angiotensin II group (−1.56±1.793) compared to the placebo group (−0.78±1.387), whereas the change in total SOFA score was not (angiotensin II: 1.35±6.032; placebo: 2.86±5.611; P=0.07).

TABLE 17
Change in CV and Total SOFA by Renin Status and Treatment
Assignment Hour 48 Change from Baseline in CV SOFA
and Total SOFA for Elevated Renin Population

| | Placebo | LJPC-501 |
|---|---|---|
| CV SOFA: N, Mean (SD) | N = 73, −0.78 (1.387) | N = 55, −1.56 (1.793) |
| Wilcoxon rank test p = 0.01 | | |
| Total SOFA: N, Mean (SD) | N = 73, 2.86 (5.611) | N = 54, 1.35 (6.032) |
| Wilcoxon rank test p = 0.07 | | |

Hour 48 Change from Baseline in CV SOFA and Total SOFA for Low Renin Population

| | | |
|---|---|---|
| CV SOFA: N, Mean (SD) | N = 63, −1.81 (1.749) | N = 64, −1.77 (1.716) |
| Wilcoxon rank test p = 0.77 | | |
| Total SOFA: N, Mean (SD) | N = 63, −0.84 (4.274) | N = 62, 0.85 (5.524) |
| Wilcoxon rank test p = 0.12 | | |

TABLE 18
Summary of Renin Samples Available at Baseline and Hour 3

| Placebo | | Angiotensin II | Total |
|---|---|---|---|
| Total Population | 158 | 163 | 321 |
| Baseline Sample | 136 | 119 | 255 |
| | 18 samples unavailable | 37 samples | 55 samples |
| | 1 samples QNS | unavailable 3 samples | unavailable 4 |
| Hour 3 Sample | 109 | 91 | 200 |
| | 40 samples unavailable | 67 samples | 107 samples |
| | 9 samples QNS | unavailable 5 samples | unavailable 14 |
| Both Baseline and Hour 3 | 108 | 90 | 198 |

QNS = quantity not sufficient

### Adverse Events

In patients with elevated renin concentrations at baseline, there was no significant difference in adverse events or serious adverse events between the angiotensin II and placebo groups.

### Discussion

In this post hoc analysis of patients enrolled in the ATHOS-3 study, it was tested whether there is a disturbance in the RAAS likely resulting from deficient ACE activity in CRVS. Such ACE deficiency could be identified through the analysis of serum renin levels, and increased serum renin levels would predict worse outcome. Serum renin levels were significantly elevated in most patients with CRVS and that were positively and significantly associated with angiotensin I levels and angiotensin I/II ratios. This ratio, as surrogate for ACE activity, is associated with increased mortality risk in CRVS. As with those patients having elevated angiotensin I/II ratios, patients with serum renin levels above the study population median had a significantly increased risk of mortality. However, this risk was attenuated by treatment with synthetic human angiotensin II. These data suggest that renin has the potential to be used to identify CRVS patients at high risk for poor outcome and who may benefit from treatment with synthetic angiotensin II.

ACE inhibition induced by drugs (e.g. captopril, enalapril) increases the levels of not only angiotensin I but of vasodilatory angiotensin I metabolites, such as angiotensin 1-7. As the absolute levels of angiotensin II do not decrease significantly during ACE inhibitor therapy, and may actually increase, the hypotensive effects of ACE inhibitors may occur as a consequence of the activity of these vasodilatory angiotensin metabolites. The similar impact of shock on angiotensin I and II levels in the present analysis suggests that ACE dysfunction resulting from endothelial damage may be a significant contributor to the pathophysiology in some patients with CRVS.

This study has direct implications for the management of patients with CRVS because renin assays are widely available and inexpensive. Thus, utilization of this biomarker for vasopressor-targeted therapy is logistically feasible. In this regard, our findings imply that utilizing angiotensin II as a vasopressor, which also lowers renin levels may offer a combined biological and clinical target during angiotensin II administration. Finally, the findings that both renin and angiotensin I are suppressible by exogenous angiotensin II suggests that patients with elevated renin may have insufficient activation of their angiotensin type I receptor.

### Conclusions

In populations with CRVS, there is significant disturbance in the RAAS likely resulting from impairment of ACE function. RAAS disturbance may play a significant role in the pathophysiology of vasodilatory shock in a subset of patients, suggesting that these patients should be managed using alternative approaches and may benefit from intervention targeting the RAAS. Importantly, patients with RAAS disturbance can be readily identified through simple laboratory assessment of serum renin levels and that they do indeed have improved outcomes when receiving synthetic angiotensin II. Our data further suggest that renin assessment could be used to identify patients without RAAS disturbance, in whom treatment with angiotensin II would likely be futile. This study suggests that a personalized approach to the management of vasodilatory shock may soon be feasible and could potentially improve outcomes in vasodilatory shock. In a setting where mortality rates can reach or even exceed 50%, such an approach is urgently needed.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control. While specific aspects of the patient matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

What is claimed is:

1. A method of treating hypotension in a human patient, comprising measuring a renin level in the patient, thereby obtaining an initial renin level; and if the initial renin level is at least a threshold value, administering to the patient angiotensin II at an initial rate.

2. The method of claim 1, wherein the threshold value is 50 pg/mL.

3. The method of claim 1, wherein the threshold value is 100 pg/mL.

4. The method of claim 1, wherein the threshold value is 150 pg/mL.

5. The method of claim 1, wherein the threshold value is 200 pg/mL.

6. The method of claim 1, wherein the threshold value is 300 pg/mL.

7. The method of any one of claims 1 to 6, wherein the initial rate of angiotensin II is at least 0.1 ng/kg/min of angiotensin II.

8. The method of any one of claims 1 to 7, wherein the initial rate of angiotensin II is at least 0.5 ng/kg/min of angiotensin II.

9. The method of any one of claims 1 to 8, wherein the initial rate of angiotensin II is at least 1 ng/kg/min of angiotensin II.

10. The method of any one of claims 1 to 9, wherein the initial rate of angiotensin II is at least 2.5 ng/kg/min of angiotensin II.

11. The method of any one of claims 1 to 10, wherein the initial rate of angiotensin II is at least 5 ng/kg/min of angiotensin II.

12. The method of any one of claims 1 to 11, wherein the initial rate of angiotensin II is at least 10 ng/kg/min of angiotensin II.

13. The method of any one of claims 1 to 12, wherein the initial rate of angiotensin II is at least 20 ng/kg/min of angiotensin II.

14. The method of any one of claims 1 to 8, wherein the initial rate is less than 1 ng/kg min.

15. The method of any one of claims 1 to 9, wherein the initial rate is less than 2.5 ng/kg/min.

16. The method of any one of claims 1 to 10, wherein the initial rate is less than 5 ng/kg/min.

17. The method of any one of claims 1 to 11, wherein the initial rate is less than 10 ng/kg/min.

18. The method of any one of the preceding claims, wherein the human patient has acute kidney injury.

19. The method of any one of claims 1 to 17, wherein the human patient has acute respiratory distress syndrome (ARDS).

20. The method of any one of claims 1 to 17, wherein the human patient has hepatorenal syndrome.

21. The method of any one of claims 1 to 17, wherein the human patient has cirrhosis.

22. The method of any one of claims 1 to 17, wherein the patient has sepsis, septic shock, distributive shock, or cardiogenic shock.

23. The method of any one of claims 1 to 21, wherein the patient does not have sepsis, septic shock, distributive shock, or cardiogenic shock.

24. The method of claim 23, wherein the patient is at risk for developing sepsis, septic shock, distributive shock, or cardiogenic shock.

25. A method of treating acute kidney injury in a human patient, comprising measuring a renin level in the patient, thereby obtaining an initial renin level; and if the initial renin level is at least a threshold value, administering to the patient angiotensin II at an initial rate.

26. A method of treating acute respiratory distress syndrome in a human patient, comprising measuring a renin level in the patient, thereby obtaining an initial renin level; and if the initial renin level is at least a threshold value, administering to the patient angiotensin II at an initial rate.

27. A method of treating hepatorenal syndrome in a human patient, comprising measuring a renin level in the patient, thereby obtaining an initial renin level; and if the initial renin level is at least a threshold value, administering to the patient angiotensin II at an initial rate.

28. A method of treating cirrhosis in a human patient, comprising measuring a renin level in the patient, thereby obtaining an initial renin level; and if the initial renin level is at least a threshold value, administering to the patient angiotensin II at an initial rate.

29. A method of treating shock in a human patient, comprising measuring a renin level in the patient, thereby obtaining an initial renin level; and if the initial renin level is at least a threshold value, administering to the patient angiotensin II at an initial rate.

30. A method of suppressing a renin level in a human patient in need thereof, comprising measuring a renin level in the patient, thereby obtaining an initial renin level; and if the initial renin level is at least a threshold value, administering to the patient angiotensin II at an initial rate.

31. The method of claim 30, wherein the human patient has a MAP of at least 55 mm Hg.

32. The method of claim 30, wherein the human patient has a MAP of at least 65 mm Hg.

33. The method of any one of claims 30 to 32, wherein the patient does not have hypotension, shock, sepsis, ARDS, AKI, hepatorenal syndrome, or cirrhosis.

34. The method of claim 30, wherein the patient is afflicted with hypotension, shock, sepsis, ARDS, AKI, hepatorenal syndrome, or cirrhosis.

35. The method of any one of claims 30 to 33, wherein the patient is at risk for developing hypotension, shock, sepsis, ARDS, AKI, hepatorenal syndrome, or cirrhosis.

36. The method of any one of claims 25 to 35, wherein the threshold value is 50 pg/mL.

37. The method of any one of claims 25 to 35, wherein the threshold value is 100 pg/mL.

38. The method of any one of claims 25 to 35, wherein the threshold value is 150 pg/mL.

39. The method of any one of claims 25 to 35, wherein the threshold value is 200 pg/mL.

40. The method of any one of claims 25 to 39, wherein the initial rate of angiotensin II is at least 0.1 ng/kg/min of angiotensin II.

41. The method of any one of claims 25 to 40, wherein the initial rate of angiotensin II is at least 0.5 ng/kg/min of angiotensin II.

42. The method of any one of claims 25 to 41, wherein the initial rate of angiotensin II is at least 1 ng/kg/min of angiotensin II.

43. The method of any one of claims 25 to 42, wherein the initial rate of angiotensin II is at least 2.5 ng/kg/min of angiotensin II.

44. The method of any one of claims 25 to 43, wherein the initial rate of angiotensin II is at least 5 ng/kg/min of angiotensin II.

45. The method of any one of claims 25 to 44, wherein the initial rate of angiotensin II is at least 10 ng/kg/min of angiotensin II.

46. The method of any one of claims 25 to 45, wherein the initial rate of angiotensin II is at least 20 ng/kg/min of angiotensin II.

47. The method of any one of claims 25 to 41, wherein the initial rate is less than 1 ng/kg min.

48. The method of any one of claims 25 to 42, wherein the initial rate is less than 2.5 ng/kg/min.

49. The method of any one of claims 25 to 43, wherein the initial rate is less than 5 ng/kg/min.

50. The method of any one of claims 25 to 44, wherein the initial rate is less than 10 ng/kg/min.

51. The method of any one of the preceding claims, further comprising increasing the rate of angiotensin II administration prior to obtaining a second measurement of renin.

52. The method of any one of the preceding claims, further comprising measuring the patient's renin level after a period of time, and if the patient's measured renin level is less than a threshold value, decreasing the rate of administration of angiotensin II.

53. The method of any one of the preceding claims, further comprising measuring the patient's renin level after a period of time, and if the patient's measured renin level is at least a threshold value, increasing or maintaining the rate of administration of angiotensin II.

54. The method of claim 52 or 53, wherein the threshold value is 50 pg/mL.

55. The method of claim 52 or 53, wherein the threshold value is 100 pg/mL.

56. The method of claim 52 or 53, wherein the threshold value is 150 pg/mL.

57. The method of any one of claims 1 to 56, further comprising measuring the patient's renin level after a period of time, and if the difference between the patient's initial renin level and the measured renin level is at least 50 pg/ml, decreasing the rate of administration of angiotensin II.

58. The method of any one of claims 1 to 56, further comprising measuring the patient's renin level after a period of time, and if the difference between the patient's initial renin level and the measured renin level is at least 100 pg/ml, decreasing the rate of administration of angiotensin II.

59. The method of any one of claims 1 to 56, further comprising measuring the patient's renin level after a period of time, and if the difference between the patient's initial renin level and the patient's measured renin level is less than or equal to 50 pg/ml, increasing or maintaining the rate of administration of angiotensin II.

60. The method of any one of claims 1 to 56, further comprising measuring the patient's renin level after a period of time, and if the difference between the patient's initial renin level is patient's measured renin level is less than or equal to 100 pg/ml, increasing or maintaining the rate of administration of angiotensin II.

61. The method of any one of the preceding claims, further comprising measuring the patient's mean arterial pressure after a period of time, and if the measured mean arterial pressure is at least 65 mm Hg, decreasing the rate of administration of angiotensin II.

62. The method of any one of the preceding claims, further comprising measuring the patient's mean arterial pressure after a period of time, and if the measured mean arterial pressure is below 65 mm Hg, increasing or maintaining the rate of administration of angiotensin II.

63. The method of any one of claims 52 to 62, wherein the period of time is about one hour.

64. The method of any one of claims 52 to 62, wherein the period of time is about three hours.

65. The method of any one of claims 52 to 62, wherein the period of time is about six hours.

66. The method of any one of claims 52 to 62, wherein the period of time is about 24 hours.

67. The method of any one of the preceding claims, wherein the renin level in the human patient after angiotensin administration is less than 250 pg/mL.

68. The method of any one of the preceding claims, wherein the renin level in the human patient after angiotensin administration is less than 150 pg/mL.

69. The method of any one of the preceding claims, wherein the renin level in the human patient after angiotensin administration is less than 100 pg/mL.

70. A method of suppressing PRA activity in a human patient in need thereof, comprising measuring a PRA activity level in the patient, thereby obtaining an initial PRA activity level; and if the initial PRA activity level is at least a threshold value, administering to the patient angiotensin II at an initial rate.

71. The method of claim 70, wherein the human patient has a MAP of at least 55 mm Hg.

72. The method of claim 70, wherein the human patient has a MAP of at least 65 mm Hg.

73. The method of claim 70, wherein the human patient has a MAP of at least 75 mm Hg.

74. The method of any one of claims 70 to 73, wherein the patient does not have hypotension, shock, sepsis, ARDS, AKI, hepatorenal syndrome, or cirrhosis.

75. The method of claim 70, wherein the patient is afflicted with hypotension, shock, sepsis, ARDS, AKI, hepatorenal syndrome, or cirrhosis.

76. The method of any one of claims 70 to 74, wherein the patient is at risk for developing hypotension, shock, sepsis, ARDS, AKI, hepatorenal syndrome, or cirrhosis.

77. The method of any one of claims 70 to 76, wherein the threshold value is 0.6 ng/mL/hour.

78. The method of any one of claims 70 to 76, wherein the threshold value is 1.0 ng/mL/hour.

79. The method of any one of claims 70 to 76, wherein the threshold value is 1.5 ng/mL/hour.

80. The method of any one of claims 70 to 76, wherein the threshold value is 2.0 ng/mL/hour.

81. The method of any one of claims 70 to 76, wherein the threshold value is 3.0 ng/mL/hour.

82. The method of any one of claims 70 to 81, wherein the initial rate of angiotensin II is at least 0.1 ng/kg/min of angiotensin II.

83. The method of any one of claims 70 to 81, wherein the initial rate of angiotensin II is at least 0.5 ng/kg/min of angiotensin II.

84. The method of any one of claims 70 to 81, wherein the initial rate of angiotensin II is at least 1 ng/kg/min of angiotensin II.

85. The method of any one of claims 70 to 81, wherein the initial rate of angiotensin II is at least 2.5 ng/kg/min of angiotensin II.

86. The method of any one of claims 70 to 81, wherein the initial rate of angiotensin II is at least 5 ng/kg/min of angiotensin II.

87. The method of any one of claims 70 to 81, wherein the initial rate of angiotensin II is at least 10 ng/kg/min of angiotensin II.

88. The method of any one of claims 70 to 81, wherein the initial rate of angiotensin II is at least 20 ng/kg/min of angiotensin II.

89. The method of any one of claims 70 to 83, wherein the initial rate is less than 1 ng/kg min.

90. The method of any one of claims 70 to 84, wherein the initial rate is less than 2.5 ng/kg/min.

91. The method of any one of claims 70 to 85, wherein the initial rate is less than 5 ng/kg/min.

92. The method of any one of claims 70 to 86, wherein the initial rate is less than 10 ng/kg/min.

93. The method of any one of claims 70 to 92, further comprising increasing the rate of angiotensin II administration prior to obtaining a second measurement of PRA.

94. The method of any one of claims 70 to 92, further comprising measuring the patient's PRA activity level after a period of time, and if the patient's measured PRA activity level is less than a threshold value, decreasing the rate of administration of angiotensin II.

95. The method of any one of claims 70 to 92, further comprising measuring the patient's PRA activity level after a period of time, and if the patient's measured PRA activity level is at least a threshold value, increasing or maintaining the rate of administration of angiotensin II.

96. The method of claim 94 or 95, wherein the threshold value is 1.0 ng/mL/hour.

97. The method of claim 94 or 95, wherein the threshold value is 2.0 ng/mL/hour.

98. The method of claim 94 or 95, wherein the threshold value is 3.0 ng/mL/hour.

99. The method of any one of claims 70 to 92, further comprising measuring the patient's PRA activity level after a period of time, and if the difference between the patient's initial PRA activity level and the measured PRA activity level is at least 0.1 ng/mL/hour, decreasing the rate of administration of angiotensin II.

100. The method of any one of claims 70 to 92, further comprising measuring the patient's PRA activity level after a period of time, and if the difference between the patient's initial PRA activity level and the measured PRA activity level is at least 0.5 ng/mL/hour, decreasing the rate of administration of angiotensin II.

101. The method of any one of claims 70 to 92, further comprising measuring the patient's PRA activity level after a period of time, and if the difference between the patient's initial PRA activity level and the patient's measured PRA activity level is less than or equal to 0.1 ng/mL/hour, increasing or maintaining the rate of administration of angiotensin II.

102. The method of any one of claims 70 to 92, further comprising measuring the patient's PRA activity level after a period of time, and if the difference between the patient's initial PRA activity level is patient's measured renin PRA activity level is less than or equal to 0.5 ng/mL/hour, increasing or maintaining the rate of administration of angiotensin II.

103. The method of any one of claims 70 to 92, further comprising measuring the patient's mean arterial pressure after a period of time, and if the measured mean arterial pressure is at least 65 mm Hg, decreasing the rate of administration of angiotensin II.

104. The method of any one of claims 70 to 92, further comprising measuring the patient's mean arterial pressure after a period of time, and if the measured mean arterial pressure is below 65 mm Hg, increasing or maintaining the rate of administration of angiotensin II.

105. The method of any one of claims 94 to 104, wherein the period of time is about one hour.

106. The method of any one of claims 94 to 104, wherein the period of time is about three hours.

107. The method of any one of claims 94 to 104, wherein the period of time is about six hours.

108. The method of any one of claims 94 to 104, wherein the period of time is about 24 hours.

109. The method of any one of the preceding claims, wherein the patient is receiving or has received an angiotensin converting enzyme inhibitor (ACE inhibitor).

110. The method of claim 109, wherein the ACE inhibitor is selected from perindopril, captopril, enalapril, lisinopril, benazepril, fosinopril, moexipril, quinapril, trandolapril, and ramipril.

111. The method of any one of the preceding claims, wherein the patient has ACE dysfunction.

112. The method of any one the preceding claims, wherein ACE activity in the patient has been inhibited.

113. The method of any one of the preceding claims, wherein the patient is receiving a vasopressor prior to administering the angiotensin II.

114. The method of claim 113, wherein the vasopressor is a catecholamine, and the catecholamine is norepinephrine, epinephrine, dopamine, or phenylephrine.

115. The method of claim 114, wherein, prior to administering angiotensin II, the patient is receiving at least 0.1 μg/kg/min of norepinephrine.

116. The method of claim 114, wherein, prior to administering angiotensin II, the patient is receiving at least 0.1 μg/kg/min of epinephrine.

117. The method of claim 114, wherein, prior to administering angiotensin II, the patient is receiving at least 5 μg/kg/min of dopamine.

118. The method of claim 113, wherein the vasopressor is vasopressin, terlipressin, argipressin, desmopressin, felypressin, lypressin, or ornipressin.

119. The method of any one of the preceding claims, wherein the angiotensin II is administered continuously for at least 12 hours.

120. The method of any one of the preceding claims, wherein the angiotensin II is administered continuously for at least 24 hours.

121. The method of any one of the preceding claims, wherein the angiotensin II is administered continuously for at least 48 hours.

122. The method of any one of the preceding claims, wherein the angiotensin II is administered continuously for at least 72 hours.

123. The method of any one of the preceding claims, wherein the angiotensin II is administered continuously for at least 5 days.

124. The method of any one of the preceding claims, wherein the angiotensin II is administered continuously for no more than 7 days.

125. The method of any one of the preceding claims, wherein the angiotensin II is 5-L-valine angiotensin II; 1-L-asparagine-5-L-valine angiotensin II; 1-L-asparagine-5-L-isoleucine angiotensin II; or 1-L-asparagine-5-L-isoleucine angiotensin II.

126. The method of claim 125, wherein the angiotensin II is 5-L-isoleucine angiotensin II.

127. The method of any one of the preceding claims, wherein the angiotensin II is in a composition, and the composition comprises angiotensin II at a concentration of about 2.5 mg/mL.

128. The method of claim 127, wherein the composition further comprises mannitol as an excipient.

129. The method of claim 128, wherein the composition comprises mannitol at a concentration of about 12.5 mg/mL.

130. The method of any one of the preceding claims, wherein the composition is administered parenterally.

131. The method of claim 130, wherein the composition is administered by injection or intravenous infusion.

132. The method of claim 130 or 131, wherein the composition is administered intravenously.

133. The method of any one of claims 1 to 132, wherein the patient is recovering from surgery.

134. A method of treating hypotension in a human patient, comprising
measuring a renin level in the patient, thereby obtaining an initial renin level; and
if the initial renin level is at least 2× the value of a normal renin level, administering to the patient angiotensin II at an initial rate.

135. The method of claim 134, wherein if the initial renin level is at least 3× the value of a normal renin level, administering to the patient angiotensin II at an initial rate.

136. The method of claim 134, wherein if the initial renin level is at least 4× the value of a normal renin level, administering to the patient angiotensin II at an initial rate.

137. The method of claim 134, wherein if the initial renin level is at least 5× the value of a normal renin level, administering to the patient angiotensin II at an initial rate.

138. The method of claim 134, wherein the normal renin level is 5 ng/ml-55 ng/ml.

139. A method of determining whether a patient with shock would be a candidate for angiotensin II therapy, comprising
measuring the a renin level in the patient,
thereby obtaining an initial renin level, and
if the initial renin level is at least a threshold value, the patient is a candidate for angiotensin II therapy.

140. The method of claim 139, further comprising administering to the patient angiotensin II at an initial rate.

141. The method of claim 139 or 140, wherein the threshold value is 100 pg/mL.

142. The method of claim 139 or 140, wherein the threshold value is 150 pg/mL.

143. The method of claim 139 or 140, wherein the threshold value is 200 pg/mL.

144. The method of claim 139 or 140, wherein the threshold value is 300 pg/mL.

145. The method of claim 139 or 140, wherein the threshold value is 3× the value of a normal renin level.

146. The method of claim 139 or 140, wherein the threshold value is 3× the value of a normal renin level.

147. The method of claim 139 or 140, wherein the threshold value is 3× the value of a normal renin level.
